(19) United States
(12) Patent Application Publication
(10) Pub. No.: US 2022/0273919 A1
(43) Pub. Date: Sep. 1, 2022

(54) INTRAVAGINAL DEVICES, COMPOSITIONS AND METHODS TO TREAT ATROPHIC VAGINITIS

(71) Applicant: PhotoKinetix Holdings Inc., Galveston, TX (US)

(72) Inventor: Marie A. Kraft, Galveston, TX (US)

(21) Appl. No.: 17/630,086

(22) PCT Filed: Jul. 24, 2020
(86) PCT No.: PCT/US2020/043369

§ 371 Date: Jan. 25, 2022

Related U.S. Application Data
(60) Provisional application No. 62/879,318, filed on Jul. 26, 2019.

(51) Int. Cl. A61M 31/00; A61N 5/06

(52) U.S. Cl. CPC A61M 31/002; A61N 5/0603; A61N 2005/0653; A61N 2005/0611

(57) ABSTRACT

The invention described herein provides devices, compositions and methods to treat atrophic vaginitis and other conditions of the vagina using combinations of vaginally applied topical therapies with light emitting devices to relieve symptoms and causations of atrophic vaginitis.

### CROSS-REFERENCE TO RELATED APPLICATION

This application claims priority to U.S. Provisional Application No. 62/879,318, filed on Jul. 26, 2019, the entire contents of which, including all drawings filed therewith, are incorporated by reference herein.

### FIELD OF THE INVENTION

The subject matter of the instant invention relates generally to treatment of the causation and symptoms associated with atrophic vaginitis. Particularly and not exclusively, the subject matter of the instant invention is related to a combinational therapeutic method comprising topically applied biologically active formulations and light emitting devices irradiating intra-vaginal topically applied formulations for the treatment of symptoms associated with atrophic vaginitis and underlying causes of vaginal genitalia mucosal tissue and cutaneous atrophy including vaginal atrophy, vulva-genital skin aging, vulvovaginal atrophy, urogenital atrophy, urinary incontinence, vaginal stenosis of the vaginal canal and vaginal opening, vaginal dryness and painful sexual intercourse.

### BACKGROUND OF THE INVENTION

Atrophic vaginitis (herein also including vaginal atrophy, vulvovaginal atrophy, and urogenital atrophy and vaginal dryness) is a common condition affecting 45% of postmenopausal women. The condition is caused by an estrogen deficiency. Estrogen levels can fall for several other reasons, including but not limited to childbirth, breast-feeding, cancer therapy (including radiation therapy, hormone therapy and chemotherapy), surgical removal of the ovaries (oophorectomy), immune disorders and stress. Furthermore, cigarette smoking and drug side effects from antihistamines and antidepressants, as well as other drug classes may also cause atrophic vaginitis and vaginal dryness. Frequent vaginal symptoms of atrophic vaginitis and vaginal atrophy include symptoms such as dryness, itching, pruritus (intense inching), leukorrhea (thick vaginal discharge), dyspareunia (painful sexual intercourse), urethral caruncle (urethral lesions), a frail, unrugated vagina (absent normal ridges and folds of the vagina) and finally vaginal stenosis and introital contraction (a narrowed and contracted introitus opening of the vagina and atrophy of exterior genitalia). In addition to changes within the internal vaginal membrane, estrogen deficiency also causes atrophy of external skin surfaces, inducing noticeable structural manifestations as evidenced vulvovaginal atrophy manifested by dryness, irritation, soreness and dyspareunia with urinary frequency, urgency, and urge incontinence.

Estradiol, the main estrogen hormone produced by the ovaries, contributes in keeping the vaginal tissues lubricated and healthy. From puberty, estrogen stimulation maintains a thick vaginal epithelium aiding the production of glycogen. Lactobacilli metabolism depends on glycogen from sloughed vaginal epithelial cells, while lactic acid produced by lactobacilli bacteria maintains a normal vaginal pH between 3.5 and 4.5. The vaginal acidic pH protects against invasion of opportunistic bacteria and fungi preventing vaginal and urinary tract infections. Estrogen insufficiency and vaginal dryness decrease the prevalence of glycogen-rich cells, causing inhibition of normal vaginal lactobacilli flora. The reduced flora further reduces the available lactic acid, and accordingly increases vaginal pH to more optimum ranges for opportunistic bacterial infection. The failure of this protective lactobacilli flora barrier causes leukorrhea, (vaginal discharge) due to contamination of sloughed epithelium by rectal and other skin resident bacterial flora.

Estrogen levels drop after menopause, causing the vaginal tissue to become atrophic (i.e., thin, dry, and shrunken). As vaginal lubricating secretions decline, sexual activity may be painful. Urinary tract epithelia are also estrogen dependent, therefore, urinary tract symptoms, such as dysuria (painful urination), and stress incontinence, may also emerge. These physiological changes within the vagina increase the likelihood of tissue trauma, infection, and can lead to dyspareunia (painful sexual intercourse). Progressively, the vaginal surface becomes friable and delicate, with petechiae (minor surface hemorrhages and bruising), ulcerations and tears with bleeding occurring from minor physical contact within the vagina. The vaginal changes from atrophic vaginitis, vaginal atrophy, and vaginal dryness may cause dyspareunia (difficult or painful sexual intercourse), leading to coitus avoidance, and can ultimately culminate in vaginal introital contraction (narrowing of the vagina opening) and rigidity of the vaginal tissues.

Cigarette smoking may also exacerbate vaginal atrophy. Smoking has a direct effect on the vaginal squamous epithelium, reduces estrogen bio-availability within the tissues, and diminishes blood perfusion throughout the vagina. Vaginal epithelium becomes thin and pale with fewer rugae (ridges and folds), with an accompanying progressive loss of vascularity and blood supply to the vaginal mucosa further compounding symptoms. Vaginal dryness can also occur as a result of medication side effects from antihistamines, benzodiazepines, beta-blockers, calcium channel blockers, medroxyprogesterone, as well as birth control medications containing ethinyl estradiol/norgestimate combination and ethinyl estradiol/norgestrel combination.

Atrophic vaginitis, vaginal atrophy and vaginal dryness symptoms and consequences can cause years of progressive vulvovaginal discomfort, with a significant impact on the quality of life for postmenopausal women. There is a positive link between sexual activity and maintenance of vaginal elasticity and pliability, in addition to the lubricative response to sexual stimulation. As the frequency of coitus and sexual stimulation diminishes, vaginal lubrication declines further. Ironically, postmenopausal women who regularly participate in the sexual activity report fewer symptoms of atrophic vaginitis and have less evidence of vaginal atrophy compared with sexually inactive women. This may be due to the sexually active women group having fewer symptoms in general, so dyspareunia is not a controlling factor, while women that have pain during sex simply avoid further painful sex. In any event, avoidance of sexual intercourse may further exacerbate vaginal atrophy symptoms. Atrophic vaginitis, vaginal atrophy, and vaginal dryness may occur because of a combination of several factors including stress, medication side effects, not only as a result of decreased estrogen.

Unlike vasomotor symptoms that typically accompany menopause, atrophic vaginitis symptoms do not diminish over time and are unlikely to resolve without treatment. Additionally, symptoms will remain under conditions wherein the root cause is a drug side effect or cigarette smoking or other somewhat discretionary causative functions i.e., taking drugs with vaginal drying side effects. Vaginal dryness increases the physiological conditions within the vagina and further causes more severe dryness with increased severity of symptoms and pathological changes within the vagina. Without some form of treatment, atrophic vaginitis and vaginal dryness are likely to progress further with increased severity of symptoms and manifestations.

Despite its high incidence, urogenital atrophy is an underreported and under diagnosed condition. Few women seek medical attention for vulvovaginal symptoms, often because they are uncomfortable talking about such a private issue or simply accept the symptoms as a condition of aging or personal circumstance. An estimated 10%-40% of postmenopausal women and 10%-25% of women receiving systemic hormone therapy experience urogenital atrophy.

General atrophic vaginitis, vaginal atrophy and vaginal dryness symptoms occur in about 47% of postmenopausal women aged 50-79 years. A further break down of specific symptoms prevalence are as follows: vaginal or genital dryness, 27%; vaginal or genital irritation, 18.6%; vaginal or genital discharge, 11.1%; dysuria, 5.2%. Of the 43% Women at age 61 years that experience vaginal dryness, 10% also experience burning. There have also been reports that 3% of women of reproductive age are troubled by vaginal dryness, and the incidence increases to 4% during the early menopausal transition, 21% in the late menopausal transition. With the current life expectancy, women could expect to spend about a full third of their life after menopause. Furthermore, more severe symptoms of vaginal atrophy have been shown to manifest in women who have never given birth vaginally compared to women who have had vaginal births.

Hormone replacement therapy (estrogen) using systemic delivery methods such as transdermal patches, transdermal gels, orally administered pills and vaginally administered suppositories have all been shown to be effective in treating and reversing symptoms of atrophic vaginitis. Vaginally applied hormone-containing creams (Estrace® Warner Chilcott), with or without concurrent system hormone replacement therapy has also been shown to be effective. Furthermore, trans-vaginal hormone application by drug releasing vaginal reservoirs such as the Estring® (estradiol, Pfizer, US) and Femring® (estradiol acitate-Warner Chilcott, Larne, U.K.) have also been shown to be effective. However, hormone replacement therapy in general may be contraindicated in women who have a history or family history of breast cancer, ovarian cancer, endometrial (uterine) cancer, history of blood clots or stroke, experience vaginal bleeding or who smoke. Hormone replacement therapy is also contraindicated in pre-menopausal women who are pregnant or want to become pregnant. Adverse side effects of hormone replacement therapy include: breast soreness/tenderness, nausea, abdominal cramping, headaches, fluid retention/bloating, weight gain, mood swings, vaginal bleeding/spotting as well as irregular skin spotting/discoloration particularly on the face. In rare but notable instances there may be an increase in uterine fibroids and a worsening of endometriosis. Other potential side effects from hormone replacement therapy are; increased risk of heart attack, stroke, breast cancer, blood clots and increased risk of dementia in women over 65 years old. Due to the potential severe adverse side effects, some women, even those with significant atrophic vaginitis symptoms, are particularly reluctant and averse to use estrogen hormone replacement therapy to treat atrophic vaginitis, vaginal atrophy and vaginal dryness symptoms.

Over-the-counter personal lubricates are frequently used to minimize friction and pain during sexual intercourse. Generally, personal lubricates marketed should be condom compatible. Lubricants containing oils should be avoided as they may weaken latex condoms. Many over the counter personal lubricants contain glycerin and/or propylene glycol. Studies have shown that there is a significant risk of yeast (vaginal candidiasis) and other infections as a result of vaginally applied glycerin and mineral and plant oils. Generally personal lubricates are effective for a short time period and are primarily applied to the vaginal opening immediately before sexual activity as a method to lubricate the vagina for sexual intercourse. Some marketed over-the-counter personal lubricants have been shown to be cytotoxic to vaginal epithelium due to formula anti-bacterial preservative and ingredient combinations. Long-term and repeated use may contribute to significant harm to vaginal tissue. See Cunha A R, et al. Characterization of commercially available vaginal lubricants: a safety perspective. Pharmaceutics, 6:3 (2014) 530-542; Dezzutti C S, et al. Is Wetter Better? An Evaluation of Over-the-Counter Personal Lubricants for Safety and Anti-HIV-1 Activity. PloS one, 7:11 (2012), e48328.

Longer-term treatment strategies that address rejuvenating vaginal tissues include vaginally instilled topical emollients. Replens® emollient containing water/oil mixtures in combination with polycarbophil and carbomer. The mode of action depends on the polycarbophil and carbomer adhering to the vaginal walls and trapping moisture within the tissues. The emollient oils such as glycerin, mineral oil and hydrogenated palm oil provide further moisturization. Glycerin and oils may contribute to vaginal yeast infections (vaginal candidiasis). Some women have reported emollient use is messy and part of the administered dose discharges from the vagina after administration. Product use instructions suggest that application should be avoided 24 hours prior to sexual contact which may be a deterrent for normal and spontaneous sexual activity. It is preferable to have an instilled vaginal topical therapy dosage that enhances normal and spontaneous sexual activity by limiting the bulk volume of the instilled dosage while providing vaginal therapeutic and lubrication function without excessive vaginal discharge.

The osmolarity of the topically applied vaginal treatments may impact the vaginal non-keratinized epithelial cellular tissue. Normal or neutral osmolarity of the normal healthy human body vagina, expressed as milliosmol per kilogram (mOsm/kg), is about 380±30 mOsm/kg. Normal and/or neutral osmolarity exerts little force across a membrane that would shift intracellular water content. Conversely, a hyperosmolar composition (compositions with an osmolarity of greater than 410 mOsm/kg) placed on the vaginal tissue would cause a water shift from the intracellular tissue into the extracellular space therefore, dehydrating the intracellular environment and changing the concentration of the intracellular constituents. See Ayehunie S, et al. Hyperosmolal vaginal lubricants markedly reduce epithelial barrier properties in a three-dimensional vaginal epithelium model. Toxicology Reports, 5 (2018) 134-140.

Additionally, hyperosmolar composition (compositions with an osmolarity of <260 mOsm/kg) placed on the vaginal tissue would cause a water shift from the extracellular tissue space into the intracellular space therefore causing the intracellular water content to increase also altering the normal concentration of the intracellular constituents. Hyperosmolar, as well as hypoosmolar extracellular conditions, can be toxic to reproducing vaginal tissue cells (cytotoxic) depending on the resulting intracellular water content after exposure to the hypoosmolar or hyperosmolar topical treatment. Vaginal moisturizers and lubricants containing glycerin and/or propylene glycol may be hyperosmolar and therefore toxic to the vaginal tissue especially when compositions also contain parabens or phenoxyethanol antibacterial preservatives. See Cunha; Dezzutti supra. Ideally, normal or near normal osmolar (380 mOsm/kg) vaginally applied topical compositions are preferable to avoid fluid shift cytotoxic effects in the vaginal tissues. In general, vaginally applied topical compositions should have an osmolarity of less than 1000 mOsm/kg and more preferably closer to the normal vaginal osmolarity of about 380±30 mOsm/kg.

The ideal vaginal lubricant with a pH of about 3.8 to 4.5 and should not exceed an osmolarity of about 380 mOsm/kg. Vaginal lubricants and topical therapies differ from the ideal lubricant intended for anal tissue therapies. Similarly, pH and osmolarity of lubricants for anal administration should be close to the normal pH and osmolarity of the rectum. The pH of the human rectum is about 7.0. To avoid tissue damage and inflammation, lubricants and therapeutics intended for anal administration should ideally have a pH of about 5.5 to 7.0 as well as have an osmolarity close to the physiological rectal osmolarity of about 285 to 310 mOsm/kg. Treating conditions of the rectum including hemorrhoids, complications of anal stenosis after surgical removal of hemorrhoids (hemorrhoidectomy), treatment of fistulas, and/or abscesses of the rectum is anticipated by the invention. In the case of anal administration, the topical therapy would be adjusted to the more ideal pH range of about 5.5 to 7.0 and an osmolarity closer to the rectal physiological range of about 285 to 310 mOsm/kg. The invention anticipates the use of a tapered anal dilator and pulse expandable device configurations in combination with topical therapy to treat conditions of the rectum.

Generally, water-based formulations to be topically applied on tissue surfaces are not manufactured under sterile conditions and usually contain some form of antimicrobial preservative compound to prevent the unwanted contamination from bacteria, fungus, and yeasts. The amount and type of preservatives are selected based on the pH of the formulation and the predicted bacterial load produced during manufacture as well as the bacterial load presented after repeated exposures after the package is opened and used by the consumer. Acidic pH formulations (pH<4.5) require less preservative concentration than formulations manufactured at a pH more agreeable for bacterial growth (about pH 7.0). Unit dose packaging wherein the product container is opened and the contents are used one-time reduces the secondary contamination potential introduced by the end user from repeated hand to container bacterial transfer. Conversely, topical products in multi-dose packaging, wherein the end user may have repeated contact with the packaged product and the heavily bacterially laden tissue surfaces of the perineum or other skin surfaces, may require significantly higher concentrations of antimicrobial preservatives to prevent contamination of the unused bulk product.

Several antimicrobial preservatives commonly used in vaginal lubricants, anal lubricants and tissue moisturizers and conditioners, as well as topical skin care products, have been shown to be cytotoxic. Parabens (esters of para-hydroybenzoic, i.e. methylparaben, ethylparaben, propylparaben, butylparaben, heptylparaben) and/or phenoxyethanol are commonly found in these products. The antibacterial mode of action is not well understood. They are thought to act by disrupting membrane transport processes or by inhibiting synthesis of DNA and RNA or of some key enzymes, such as ATPases and phosphotransferases. The cytotoxicity tends to be concentration dependent. A significant synergistic increased cytotoxicity is produced when these preservatives are in combination with hyperosmolar formulations. It is preferable that vaginal lubricants and moisturizers, as well as topical skin care products and anal lubricants and therapies, do not contain cytotoxic substances that are harmful to tissue cells. Also, the use of high concentration preservatives with wide antimicrobial action may interfere with the beneficial normal resident bacterial flora within the vagina and may then prevent the restoration of normal bacterial flora conditions within the vagina. Antimicrobial preservatives, while necessary for product safety, should be selected to have minimal harmful effects on tissues. Product antimicrobial preservatives should be incorporated at the lowest effective concentrations in products used in unit dose packaging. Alternatively, compositions can be comprised of non-toxic bioactive compounds that have a secondary function of composition antimicrobial preservation without being cytotoxic to cells of the vaginal membrane tissues.

Hyaluronic Acid and its derivative sodium hyaluronate have been used in cosmetic formulations to provide a hydrating film onto the skin to trap moisture. Recently, Chen et al. published a clinical trial showing that a formulation “Hyaluronic acid vaginal gel” (Hyalofemme®, Fidia Farmaceutici S.p.A., Abano Terme, Italy) comprised of a compound derived from hyaluronic acid (Hydeal-DTM®) was equivalent to a hormonal estradiol cream preparation in reducing symptoms of atrophic vaginitis. See Chen J et al. Evaluation of the efficacy and safety of hyaluronic acid vaginal gel to ease vaginal dryness: a multicenter, randomized, controlled, open-label, parallel-group, clinical trial. J Sex Med 10:6 (2013) 1575-1584. Several vaginal dryness preparations containing hyaluronic acid are now marketed including: Hyalofemme®. Some of these products may contain glycerin, mineral and plant oils and may have associated potential for cytotoxic hyperosmolar fluid shifts and/or the potential for promotion of vaginal yeast infections (genital candidiasis). Additionally, the combination of hyperosmolar ingredients including glycerin and or propylene glycol, along with formula antimicrobial such as phenoxyethanol and or parabens or combinations of parabens (methylparaben, ethylparaben, propylparaben, and butylparaben) may be cytotoxic to vaginal tissues. See Cunha; Dezzutti supra.

External skin moisturization formulations commonly contain glycerin, mineral and plant oils. However, there are some water-soluble constituents in these formulations that are notable for providing skin hydration and structural restorative effects (collagen and elastin production stimulation) that are suitable for application within the vagina. These include sodium hyaluronate, 1,2-propanediol, aminopropyl ascorbyl phosphate, tocopherol acetate, panthenol, niacinamide, L-Carnitine and collagen production stimulating peptides (GHK (tri-peptide), pentapeptide palmitoyl pentapeptide-3, palmitoyl pentapeptide-4, amphiphile (PA) C-KTTKS), stem cell growth medias and others. The invention anticipates the composition incorporation of peptide/protein based human stem cell growth media.

Because of the reluctance to use, the inherit physiologic risks and the side effects of hormone replacement therapy to treat conditions and symptoms of atrophic vaginitis, vaginal atrophy and vaginal dryness, a need exists for a safe and effective non-hormonal therapy to alleviate, mitigate, reverse and/or treat atrophic vaginitis, vaginal atrophy and vaginal dryness. It would therefore be desirable to provide compositions, methods and apparatuses to address these problems.

### SUMMARY OF THE INVENTION

The instant invention provides a combinational therapeutic method for the treatment of symptoms associated with atrophic vaginitis and underlying causes of vaginal genitalia mucosal tissue and cutaneous atrophy, wherein the method includes the combination of vaginally topically applied biologically active formulations and virginally inserted light emitting devices irradiating topically applied formulations on the vaginal surface, thereby facilitating the vaginal interstitial deposition of topically applied/instilled biologically active substances as topical vaginally applied compositions.

Principally, in accordance with a primary embodiment of the present invention, the therapeutic method includes a photokinetic intravaginal trans-membrane interstitial delivery of a biologically active composition including the steps of: applying a formulation comprising the biologically active substance to the non-keratinized cellular surface within the vagina and onto vaginal tissues of an adult human female; illuminating the formulation on the vaginal cellular surface with a pulsed light having a selected wavelength, selected pulse rate and selected duty cycle wherein the pulsed light originates from a light source comprised in a vaginally insertable irradiating device inserted in the vagina of the adult human female and allowing and/or enabling the biologically active substance in the formulation to permeate through said cellular surface of the mucus membrane of the human female vagina, thereby effecting light facilitated intra-vulvovaginal, intradermal, intravaginal, intramucosal, and/or interstitial deposition of the biologically active substance formulation in interstitial concentrations exceeding the achievable interstitial concentrations by passive concentration gradient topical application.

Furthermore, the invention provides the targeted delivery of the biologically active substance into vaginal tissues. The targeted delivery prevents the damages to the vaginal tissue and further prevents the unpredictable effects resulting from the non-targeted systemic absorption of the biologically active substance.

The biologically active composition is comprised in a pharmaceutically acceptable composition form suitable for the application onto the vaginal tissue surfaces and supporting the vaginal flora. The biologically active composition on the tissue surface available for translocation from the tissue surface into the vaginal tissue interstitial space is facilitated by the selected pulsed light irradiation. The translocation from the vaginal tissue surface into the vaginal tissue enables the creation of a pathway for enhanced drug tissue deposition without causing damage to the vaginal tissue membrane of the vaginal tissue. The light energy actuating the biologically active compound translocation; further reversibly excites the biologically active molecules in the composition with selected light energy thereby preventing the transformation or chemically degrading or photochemically denaturing of the biologically active molecules. The targeted delivery further prevents the chemical alteration and transformation of active ingredients included in the biologically active substance composition. The rate of delivery of the biologically active composition is regulated by modulating at least one characteristic of the pulsed light pulse frequency, pulse duration, light flux power and or selected light wavelength. The biologically active substance(s) in the topically applied composition preferably have a molecular weight in the range of about 165 Dalton (Da) to about 2.4 kilo Dalton (kDa).

The formulation composition is selected from the group consisting of biologically active compounds and biologically active compounds combined with other topical formula constituents. The formulation composition is further selected from the group consisting of: a solution and a solvent, and/or a hormone and/or a gelling agent, and/or a chemical, and/or an emollient system, and/or a drug, and/or a chemical, and/or a peptide, and/or an active compound, and/or deoxyribonucleic acid (DNA), and/or ribonucleic acid (RNA), and/or an emulsified biologically active substance.

Additionally, in accordance with an embodiment of the present invention, the therapeutic composition biologically active constituents are selected to provide vaginal tissue thickening, conditioning moisturization flexibility and compliance.

Additionally, in accordance with an embodiment of the present invention, the therapeutic method includes a vaginally insertable therapeutic light device having a plurality of electronic circuits producing pulsed electronic signals driving a plurality of the light emitting diodes (LEDs) to produce the pulsed light of selected wavelength, selected pulse rate, and selected pulse duration. The plurality of electronic circuits, the plurality of light emitting diodes (LEDs) and a power supply unit are enclosed in an internal reflecting spherical enclosure. The internal reflecting spherical enclosure is an enclosure made of a transparent or translucent silicone rubber, a transparent plastic enclosure, and/or a transparent hardened glass enclosure, wherein the material of the enclosure allows the light from the light emitting diode (LED) to reflect within the internal reflecting spherical enclosure to prevent the substantial loss in the intensity of light until the totally internally reflected light is extracted from the light source. The totally internally reflected light from the enclosure is scattered onto the tissue surface to effectuate tissue contact, facilitating the effective transportation of the light from within the vaginally inserted light device onto the biologically active substance on vaginal tissue surface.

Additionally, in accordance with an embodiment of the present invention, the therapeutic method includes a vaginally insertable therapeutic light device having a plurality of electronic circuits producing pulsed electronic signals driving layers of organic electroluminescent material (OLED) to produce the pulsed light of selected wavelength, selected pulse rate, and selected pulse duration. The plurality of electronic circuits, the layers of organic electroluminescent material and a power supply unit are enclosed in an internal reflecting enclosure directing light out of the enclosure. The organic electroluminescent material may be layered on the device surface or layered within the device covered and housed and additionally layered with a transparent material including a transparent or translucent silicone rubber, a plastic enclosure and or a transparent hardened glass enclosure, wherein the material of the enclosure allows the light from the organic electroluminescent material light to emit from the enclosure to prevent the substantial loss in the intensity of light. A signal organic electroluminescent material (OLED) can be used as the lone light source or combinations of different organic electroluminescent materials formulated to produce different emitting wavelengths, or additionally combined with traditional LED diode packages within the same vaginal insertable device; the object of the light device light emitting properties being to emit pulsed light with selected light wavelengths and selected light radiant power directed onto the vaginal tissues, and onto the biologically active substance applied to vaginal tissue surface.

In summary, the invention described herein provides devices, compositions and methods to treat atrophic vaginitis and other conditions of the vagina, using combinations of vaginally applied topical therapies with light emitting devices to relieve symptoms and causations of atrophic vaginitis. The devices of the invention are configured to provide pulsed visible and infrared light energy onto biologically active topical therapies applied to the vaginal tissues to be treated, as a method for enhanced deposition of therapies into the non-keratinized tissue structures of the vagina. The methods of the invention include the steps of applying a formulation comprising the biologically active substance to the cellular surface within the vagina and vaginal tissues of a human adult female, and irradiating the formulation on the cellular surface with a pulsed light having a selected wavelength, pulse rate and duty cycle, thereby actuating the translocation of the biologically active substance from the tissue surface into and through the non-keratinized epithelial cellular surface of the mucus membrane of the vagina effecting intra-vulvovaginal, intradermal, intravaginal, intramucosal, and interstitial deposition of the biologically active substance in the formulation, facilitating the targeted delivery of the biologically active substance into vaginal tissues. The device combination with topical therapy targeted delivery prevents thermal damages to the vaginal tissue, and prevents the unpredictable effects from the non-targeted systemic absorption of the biologically active substance. In certain embodiments, the devices of the invention are configured to provide selected light energy wavelengths, pulse rates and duty cycles. In certain embodiments, the devices of the invention are configured to allow placement within the vagina and affect light irradiation of topical therapies applied to the vaginal tissues and irradiation of internal vaginal tissues and genitalia, adjacent urogenital structures and external genitalia. In certain embodiments, the devices of the invention are configured to provide dilation of the vaginal canal and vaginal introitus to enhance vaginal tissue compliance and flexibility.

The following description is illustrative in nature, and is not intended to be in any way limiting. In addition to the illustrative aspects, embodiments, and features described above, further aspects, embodiments, and features will become apparent by reference to the following detailed description.

### BRIEF DESCRIPTION OF THE DRAWINGS

The accompanying drawings illustrate the best mode for carrying out the invention as presently contemplated and set forth hereinafter. The present invention may be more clearly understood from a consideration of the following detailed description of the illustrative embodiments, taken in conjunction with the accompanying drawings wherein like reference letters and numerals indicate the corresponding parts in various figures in the accompanying drawings.

FIG. 1 Illustrates a cross sectional view of an atrophied vagina and surrounding and adjacent tissue structures in the lower abdomen of a human female. This illustration is used with continued reference in several of the following figures.

FIG. 2 illustrates a rigid round housing of a fully insertable intravaginal light device with an array of light sources embedded in an optically clear rigid housing with electronic driver circuits electrically coupled to LEDs along with an integral power supply and induction charging system.

FIG. 3A illustrates an intravaginal light device showing an internal etching method to effectively scatter and diffuse of the light within the device thereby providing and even light diffusion for irradiating vaginal tissue.

FIG. 3B illustrates an intravaginal light device showing an additional internal etching method to effectively scatter and diffuse of the light within the device thereby providing and even light diffusion for irradiating vaginal tissue.

FIG. 3C illustrates an intravaginal light device showing an another internal etching method to effectively scatter and diffuse of the light within the device thereby providing and even light diffusion for irradiating vaginal tissue.

FIG. 4 illustrates the positioning of a fully insertable cylindrical housing rigid light device within the vagina.

FIG. 5A illustrates a side view of a fully insertable waterproof flexible, nonrigid clear silicone intra-vaginal device with internal LEDs, electronic drive circuits, battery and induction charging a components.

FIG. 5B illustrates a reverse, reflected side view of a fully insertable waterproof flexible, nonrigid clear silicone intra-vaginal device with internal LEDs, electronic drive circuits, battery and induction charging a components.

FIG. 5C illustrates a perspective view of the external surface of a fully insertable waterproof flexible, nonrigid, clear silicone intra-vaginal device shown in FIG. 5A.

FIG. 5D illustrates a reverse perspective view of the external surface of a fully insertable waterproof flexible, nonrigid, clear silicone intra-vaginal device FIG. 5B.

FIG. 6 illustrates the positioning of the flexible, nonrigid clear silicone intra-vaginal light device within the vagina as well as the positioning of the retrieval arm positioned adjacent to the external genitalia.

FIG. 7A illustrates an induction charging system housing intended to accept the fully insertable waterproof flexible, nonrigid clear silicone intra-vaginal device shown in FIG. 5B and FIG. 5D the battery charging housing.

FIG. 7B recalls the position of the induction charging coil of the intravaginal light device as shown in FIG. 5B further illustrating the proximity of the charging housing induction coil and the vaginal unit induction charging coil.

FIG. 7C illustrates a perspective view of the flexible, nonrigid clear silicone intravaginal light device positioned within the electrical induction battery charging housing.

FIG. 8A illustrates a configuration of a rigid or semi-rigid vaginal dilating device configurations with a progressively tapered cylindrical design intended to progressively dilate the atrophied vaginal opening and the vaginal canal wherein an encapsulated and potted light emitting array is positioned to project light within the dilating portion of the device.

FIG. 8B illustrate a configuration for rigid or semi-rigid vaginal dilating device configurations with a progressively tapered cylindrical design intended to progressively dilate the atrophied vaginal opening and the vaginal canal wherein an encapsulated and potted light emitting array is positioned to project light laterally from within the dilating portion of the device onto surrounding vaginal tissues.

FIG. 8C illustrates a perspective view of the external surface of configurations as shown in FIG. 8A and FIG. 8B of the vaginal dilating device configurations.

FIG. 9 illustrates the rigid or semi-rigid vaginal dilating device FIG. 8A or FIG. 8B within the vagina allowing for vaginal opening and vaginal canal dilation.

FIG. 10A illustrate a flexible pulsatile pneumatic initiated expandable vaginal dilation device in the deflated and relaxed condition.

FIG. 10B illustrate a surface perspective view of the flexible pulsatile pneumatic initiated expandable vaginal dilation device shown in FIG. 10A in the deflated and relaxed condition.

FIG. 10C illustrate a flexible pulsatile pneumatic initiated expandable vaginal dilation device in the pressurized expanded condition.

FIG. 10D illustrate a surface perspective view of the flexible pulsatile pneumatic initiated expandable vaginal dilation device shown in FIG. 10C in the pressurized expanded condition.

FIG. 11 illustrate the pneumatic dilating device in the relaxed mode (FIG. 10A) within the vagina.

FIG. 12 illustrate the pneumatic dilating device in the expanded pressurized mode (FIG. 10C) within the vagina.

FIG. 13 illustrates the pulsatile pneumatic dilating device operating parameters with pressure and vaginal dilating function of pressure and dilating dimension feedback with pneumatic pressure and resulting vaginal dilation device in the relaxed (FIG. 10A) mode and inflated function (FIG. 10C).

FIG. 14 illustrates a wireless handheld operational control and visual feedback monitor that provides numerical valuation of vaginal tissue compliance, vaginal contraction force and vaginal dilation dimensions.

### DETAILED DESCRIPTION OF THE INVENTION

### 1. Overview

The devices and methods of the invention utilize non-ablative LED (light emitting diode) therapy, phototherapy, low level laser therapy (LLLT) and/or photobiomodulation, which uses light wavelengths from about 600 nanometers to about 1100 nanometers (nm) to irradiate tissues, in order to cause a beneficial effect on human skin and wound healing by promoting collagen synthesis. Additionally, LED light therapy is used to increase collagen production within irradiated tissues, to increase tissue thickness, and to make tissues more resilient and compliant. LED light therapy photobiomodulation, as practiced in the subject invention, is used to increase collagen production within irradiated vaginal tissues increasing tissue thickness, making the vaginal wall more resilient and compliant.

It has been determined that light irradiation in the 600 to 1100 nm range does not adversely affect the normal healthy bacterial flora of the vagina; normal vaginal bacterial flora being necessary for vaginal rejuvenation and health. Conversely, light wavelengths in the ultraviolet, near ultra violet and visible blue wavelengths from about 350 to 450 nm may harm or disrupt the normal vaginal bacterial flora and should thus be avoided. Thus, the invention provides the use of light therapy wavelength range from about 600 to 1100 nm that increases collagen production within the atrophied vaginal tissue, thereby increasing thickness and structural durability, and reducing painful sexual intercourse, without harming beneficial resident bacterial flora. The use of light as a photobiomodulation component along with topically applied or instilled biologically active compositions is provided in the invention.

Additionally, the device of the invention may be coated with or otherwise supplies, or facilitates the photokinetic delivery of, intra-vaginally applied topical formulations, which may comprise drugs that have therapeutic effects for the treatment of, or additionally other than the treatment of, atrophic vaginitis, vaginal atrophy and vaginal dryness, including antivirals, anti-microbials, spermicidal, sperm motility promoters, as well as drugs delivered for system uptake. Thus, the subject invention incorporating topical therapies with light-based devices facilitate an intra-vaginal drug delivery system that is of use to modulate the delivery of these medications into vaginal tissue interstitial space.

In U.S. Pat. No. 9,474,911 B2, Kraft et al. discloses the application of pulsed incoherent light increases the permeation of 2.2 kilo Dalton (kDa) hyaluronic acid into human split-thickness skin through the intact and permeation restrictive stratum corneum keratinized surface layer. In U.S. Pat. No. 7,458,982 B2 Kraft et al. and U.S. Pat. No. 7,854,753 B2 Kraft et al. disclosed a method of enhanced intradermal deposition of various compounds with molecular weights from 168 Dalton (Da) to 5,754 Da into split-thickness human skin by the application of pulsed incoherent light. The disclosed method theorized that the pulsed light irradiation caused reversible molecular conformational changes in the exposed topically applied biologically active drug molecules that then imparted physical molecular movement as a method of enhanced delivery of topically applied biologically active compounds into and for deposition within underlying dermal tissues through the stratum corneum of the spit thickness skin.

Depending on the light wavelength, light energy irradiating biologically active substances, tissue constituent structures, and layers and compounds, as well as bound and un-bound, water on and within the interstitial space can be absorbed by molecular bonds within the constituents and/or transmitted through the constituents without light energy absorption. Herein, light wavelengths are selected specifically to be absorbed by topically applied biologically active substances, tissue constituent compounds as well as the water layer residing on the tissue membrane surface and the interstitial water within the tissue compartments. Without being bound by theory, the present invention anticipates that topically applied biologically active substances, tissue constituent compounds as well as bound and un-bound water on and within the vaginal tissue interstitial space, accept and absorb selected pulsed light energy irradiation and react by initiating molecular bond conformational changes thus cyclically actuating physical movement and/or causing molecular vibration within the irradiated entities. The cyclic actuated physical movement initiated by the application of selected pulsed incoherent light irradiating compounds positioned on the non-keratinized tissue surface (absent a cornified stratum corneum layer) induces enhanced intramucosal, interstitial deposition of the topically applied biologically active compounds into the underlying mucosal tissues and mucosal membranes of the vagina. The application of selected pulsed light parameters onto topically applied biologically active substances affects and enhances the topical substance translocation from the non-keratinized tissue surface into the underlying tissues, membranes and interstitial spaces in fluxes, rates and quantities greater than the achievable passive concentration gradient tissue permeation flux and resulting increase in tissue deposition quantities. The application of selected pulsed light parameters onto topically applied biologically active substances facilitates non-keratinized tissue deposition in greater concentrations of biologically active substances within the tissues by the application of selected light parameters onto the vaginally applied biologically active substances.

Various vaginal topically applied bioactive compounds with molecular weights from 168 Da to 2.2 kDa are thereby actuated and translocated from the vaginal tissue surface for vaginal interstitial deposition within the interstitial space of the mucosal tissues by the application of selected pulsed light irradiation with selected light wavelengths and pulse rates and pulse duty cycles directed onto the topically applied bioactive compounds irradiating said compounds and underlying vaginal tissues.

Further, oxidative stress within light irradiated tissues may be induced by light therapy itself. Compositions comprising compounds that attenuate oxidative stress within tissues are also anticipated by the invention. Vitamins and anti-oxidative compounds, for example including but not limited to Vitamin C, Vitamin E, coenzyme Q10, hydroxytyrosol, resveratrol, green tea extracts (i.e. epigallocatechin gallate) and other antioxidant polyphenols are anticipated to be incorporated in the vaginally applied topical or vaginally instilled composition.

Accordingly, the present invention overcomes the many shortcomings of the treatment methods and therapies known in the prior art for the disorders associated with atrophic vaginitis, by providing a non-toxic, topically applied/instilled biologically active compositions that provides lubrication, moisturization and/or tissue restorative properties, combined with the devices of the invention to facilitate vaginal interstitial deposition of topically applied/instilled biologically active substances as compositions for the treatment of disorders associated with atrophic vaginitis, and underlying causes of vaginal genitalia mucosal tissue and cutaneous atrophy, including vaginal atrophy, vulva-genital skin aging, vulvovaginal atrophy, urogenital atrophy, vaginal dryness and painful sexual intercourse.

In addition, physical stretching of the atrophied vagina and vaginal introitus (vaginal opening) also has a benefit. Vaginal dilators are commonly used to progressively stretch the introitus and vaginal canal as a means to prevent painful sexual intercourse. Devices that aid in vaginal dilation and compositions that aid vaginal lubrication rehabilitate and condition the atrophied vagina opening and internal vaginal canal making the opening and internal tissues more compliant and flexible thereby reducing pain during sexual intercourse. The invention anticipates therapeutic light and topical therapies with facilitated permeation light emitting components to be configured as integral parts of rigid, semi-rigid and flexible vaginal dilating devices with examples of such configurations in drawings and descriptions herein.

Physical stretching of atrophied vagina and introitus of the vagina combined with light therapy and enhance delivery of topical therapies into the atrophied tissues increase the flexibility and compliance of the structures and tissues. Increased vaginal tissue compliance and flexibility relieves pain associated with sexual intercourse. Tissue compliance is a measurement of tissue flexibility with a specified force. A therapeutic object of the invention is to increase compliance of the introitus of the vagina and the vaginal canal itself. Another object of the invention is to progressively dilate and stretch the atrophied vagina without causing prolonged intervals of tissue ischemia and associated adverse side effects of tissue reperfusion. Vaginal tissue ischemia, in this context, is the amount of pressure placed on the tissues that cause a decrease in capillary blood flow. The invention anticipates a vaginal light device configured with a transparent or semi-transparent flexible sheath that inflates and dilates at selected time intervals within the vagina to initiate tissue stretching and also provides selected time intervals of reduced dilating force using pressures less than blood capillary fill pressures as a means to reduce tissue reperfusion injury. In this illustration, a pulsatile stretching function is followed by a period of relaxation of the flexible sheath while leaving the sheath partially expanded to a vaginal space volume, however, at an absolute pressure below the capillary blood diastolic pressure thus allowing blood flow within the tissue capillaries and avoiding tissue ischemia.

Constituent with pulsatile vaginal stretching and vaginal volume expansion with a selected force; the tissue flexibility and compliance can be calculated if the vaginal volume is known under the relaxed and stretched interval condition. In one embodiment the invention anticipates a pulsatile vaginal stretching and relaxation pressure sensor function along with a volume and or proximity sensor that provides a vaginal volume value. With a known pressure value and a known vagina volume or diameter value of relaxed tissue compared to a known pressure value and a known volume or vaginal diameter value of expanded tissue, a tissue compliance value can be generated and is illustrated as a function of the equation:

$$\frac{Stretched Vaginal Dilation Volume}{Dilator Pressure} = Compliance \times Factor = Tissue Compliance Value$$

Whereas the relaxed static pressure and volume/diameter is normalized to 0 and the amount of pressure exerted to produce a certain expanded vaginal volume is generated; then a compliance value is generated.

For example, an increase in vaginal diameter above the relaxed value is 2.5 centimeters (cm) using a pressure of 150 mm of mercury (Hg). A tissue compliance value of 1.66 is generated:

$$\frac{2.5 cm}{150 mm Hg pressure} = 0 \text{.0166} \times 100 = 1.66 Tissue Compliance Value$$

With continued vaginal stretching and therapeutic treatments, the same subject attains an increase in vaginal diameter above the relaxed value of the same 2.5 centimeters (cm), however requiring a reduced pressure of 75 mm of mercury (Hg) to reach the same expanded volume. A tissue compliance value of 3.3 is generated:

$$\frac{2.5 cm}{75 mm Hg pressure} = 0.0333 \times 100 = 3.3 Tissue Compliance Value$$

In this example, less pressure is required to achieve the same vaginal volume/vaginal diameter indicating the tissue is more compliant and flexible. In certain embodiments, the invention incorporates pressure and volume sensors in a flexible and expandable dilating sheath to generate tissue compliance values. The invention also anticipates an informational feedback to the subject to demonstrate vaginal dilation treatment efficacy. In this same embodiment, the same device configuration can also be used to provide a therapeutic vaginal exercise system wherein a vaginal contraction dimension value and pressure force exerted for the contraction can provide quantification bio-feedback of the vaginal exercise contraction as a vaginal exercise and toning system.

Several aspects of the present invention are as stated below:

In one aspect, the present invention provides a combinational therapeutic method to treat a disorder pertinent to atrophic vaginitis including vaginal atrophy and vaginal dryness, the method comprising positioning an enhanced non-thermal light-based delivery device within the vagina of an adult human female, wherein a biologically active topical formulation suitable for vaginal application is vaginally applied for photokinetic delivery by the device, and/or wherein the surface of the device comprises a biologically active topical formulation suitable for vaginal application.

In general, the biologically active topical formulation can be applied: 1) directly onto the vaginal tissues before the device of the invention is inserted; 2) onto the device of the invention before the device is inserted into the vagina; or 3) onto vaginal tissue as well as onto the device of the invention. For the sake of simplicity, however, some embodiments or aspects of the invention described herein refer to only one of the three possibilities above. But each of such embodiments or aspects should be read to include all three mode of applying the biologically active topical formulation.

That is, in certain embodiments, the biologically active topical (therapeutic) composition is applied to the surface of the intravaginal device to lubricate the device and lubricate tissues to ease insertion, while also providing a method of topical application to the vaginal tissues.

In certain embodiments, the biologically active topical (therapeutic) composition is directly applied to the surface of the vaginal tissues to lubricate the tissues to ease intravaginal device insertion in addition to providing a method of topical application to the vaginal tissues.

In certain embodiments, the biologically active topical (therapeutic) composition is directly applied to the surface of the intravaginal device and additionally applied directly onto the vaginal tissues to lubricate the tissues for intravaginal device insertion in addition to providing a method of topical application to the vaginal tissues.

In certain embodiments, the method comprises inserting the device into the vagina to perform a vaginal treatment option, such as light therapy, transvaginal electrical stimulation, and/or vibration features incorporated in the device architecture, wherein the surface of the device is coated with the biologically active topical (therapeutic) composition, before the removal of the device after treatment, thereby leaving the biologically active topical (therapeutic) composition in place within the vagina.

In certain embodiments, the biologically active topical (therapeutic) composition provides tissue conditioning and/or vaginal lubrication to relieve pain and symptoms of atrophic vaginitis, and/or to enhance sexual intercourse comfortness.

Another aspect of the invention provides an intravaginal non-thermal light irradiating device, for intravaginal, intra-mucosal drug delivery into the interstitial space of the vaginal tissues, wherein the device is coated with a biologically active substance in a topical formulation suitable for vaginal application. In certain embodiments, the device is capable of delivering said biologically active substance, in interstitial deposition concentrations exceeding concentrations achieved by concentration gradient passive permeation of an applied topical formulation comprising said biologically active substance.

Another aspect of the present invention provides a method to facilitate dilation of the atrophied and stenosed vagina, the method comprising inserting a vaginal dilating device with a desired structure, size, and shape to facilitate dilation, wherein the device is coated with a biological substance in topical formulation suitable for vaginal application, and wherein the device is a light irradiating device capable of delivering said biological substance through the mucosal surface of the vagina.

Another aspect of the present invention provides a method to facilitate increased vaginal tissue compliance, the method comprising inserting into the vagina of an adult human female a light irradiating device coated with a topical formulation, wherein the device is further equipped to provide electrical stimulation, vibrational stimulation, and/or biofeedback, wherein the device provides increased or enhanced flexibility and/or tightened tone to atrophied vagina.

In certain embodiments, the device has a pre-determined structure, size, shape, and/or function to provide phototherapeutic vaginal tissue restructuring and/or vaginal muscle toning.

Yet another aspect of the present invention provides a composition, method, and/or device for photokinetic intra-vaginal delivery of a therapeutic substance into the vaginal membrane interstitial space of an adult human female, without causing damage and/or thermal injury to the membrane or underlying tissue thereof.

A further aspect of the present invention provides a composition, method, and/or device for photokinetic intra-vaginal delivery of a therapeutic substance into the vaginal membrane interstitial space of an adult human female, without denaturing and/or degrading the therapeutic substance being administered.

An additional aspect of the present invention provides a method to stimulate collagen production, to thicken vaginal walls, to rejuvenate and/or thicken vaginal tissues, to increase tissue tone, flexibility, and/or compliance of an adult human female, the method comprising applying pulsed visible red (600-700 nm), and/or near-infrared light wavelengths (700-1400 nm) onto vaginal tissues of said female, thereby stimulating collagen production, thickening the vaginal walls, rejuvenating and/or thickening vaginal tissues, increasing tissue tone, flexibility, and/or compliance.

An additional aspect of the present invention provides a method to dilate blood vessels and/or to produce increased blood flow through the tissues of an adult human female without causing tissue thermal degradation, the method comprising applying a selected pulsed visible red (600-700 nm), and/or near-infrared light wavelengths (700-1400 nm) onto vaginal tissues of the adult female, with modulated light pulse frequency, duty cycle, and light radiant power and flux to prevent thermal damage to the tissues, thereby dilating blood vessels and/or producing increased blood flow through the tissues without causing tissue thermal degradation.

Yet another aspect of the present invention provides a method for tissue rejuvenation, the method comprising applying, onto vaginal tissues of an adult female, pulsed visible light (600-700 nm) and/or near-infrared light wavelengths (700-1400 nm) that do not disrupt, kill or adversely affect normal, healthy vaginal bacterial flora, to effectuate light-facilitated interstitial biologically active substance delivery, through actuating, translocating and/or depositing a biologically active substance in a topical formulation suitable for vaginal delivery, into underlying vaginal tissue interstitial spaces.

Another aspect of the present invention provides a method to retain moisture and/or to relieve a symptom of vaginal dryness, the method comprising administering a composition comprising hyaluronic acid (HA) and a derivative thereof into vaginal tissues of an adult female in need thereof.

Yet another aspect of the present invention provides a method to incorporate tissue-humidifying and tissue conditioning substances into therapeutic compositions to be applied to the vaginal tissue, using the method and devices of the invention.

A further aspect of the present invention provides a method to incorporate compounds with a biological activity of collagen production stimulation into a therapeutic formulation to be applied to the vaginal tissue, using the method and devices of the invention.

Still further aspect of the present invention provides a method to enhance comfort during sexual intercourse, the method comprising applying a therapeutic formulation of the invention as a personal lubricate to the vaginal tissue of an adult human female.

An additional aspect of the present invention provides a method to treat or prevent a disease of the vagina, the method comprising applying a therapeutically effective amount of a topically applied formulation comprising a compound with antibacterial, antifungal, and/or antiviral activity against the disease, with the device of the invention.

Another aspect of the present invention provides a vaginally insertable light device of optimal size, shape and/or structure, for use in causing dilation of constricted atrophied vaginal opening, and/or dilation of atrophied vaginal canal.

Yet another aspect of the present invention provides a vaginally insertable light device of optimal size, shape and/or structure, for use as a treatment to cause the enlargement and enhanced tissue compliance and flexibility of atrophied and confined vaginal spaces, in order to provide enhanced comfort during sexual intercourse.

Further aspect of the present invention provides a combination therapeutic method for treating vaginal atrophy, the method comprises using the subject vaginally insertable light device to deliver a topical therapeutic composition to vaginal tissues of an adult human female, thereby reducing vaginal rigidity and/or stiffness, promoting compliantness and/or flexibility of the vaginal tissues and/or structures, and/or allowing for greater comfort during sexual intercourse.

Another aspect of the present invention provides a vaginally insertable light device, which, when inserted into the vagina, advantageously dilates rigid vaginal canal space to render the vaginal tissues more compliant and more flexible, thus allowing for an enhanced comfort during sexual intercourse.

Additional aspect of the present invention provides a combination therapy for treating urinary incontinence in an adult human female, the method comprises delivering a therapeutically effective amount of a topical therapeutic composition through using any one of the vaginally insertable light device of the invention to therapeutically treat vaginal atrophy, wherein the vaginally insertable light device is configured to have a vibrational mode of operation.

An additional aspect of the present invention provides a method to treat vaginal atrophy, the method comprising positioning a vaginally insertable light device coated with a topical therapeutic composition inside the vagina of an adult female to deliver the topical therapeutic composition, wherein the insertable light device is configured to have an electrical mode of operation to provide transvaginal electrical stimulation of vaginal muscle in order to produce cyclic vaginal canal muscle contractions to tighten the vaginal canal and pelvic floor. In certain embodiments, the method further treats urinary incontinence. In certain embodiments, the method further treats persistent vulvar pain.

In certain embodiments, the device of the invention incorporates more than one functions into the same housing of the device, including light therapy, vaginal contraction exercise, vibration and/or electrical stimulation. Such devices may be used to treat various conditions associated with vagina atrophy, as well as tissues of the urinary bladder including the internal and exterior sphincter muscles of the urethra to treat urinary incontinence.

In certain embodiments, the device further incorporates a feedback tracking system to facilitate and guide a vaginal exercise regimen, including a vaginal pelvic floor exercise.

### 2. Technical Advantages and Economical Significance of the Present Invention

The present invention provides an improvised device and method for intravaginal light therapy. The invention in accordance with the most preferred embodiment provides a combinational therapeutic method having topical formulations combined with enhanced light-based delivery to overcome the problems in treating disorders pertinent to atrophic vaginitis including vaginal atrophy and vaginal dryness. In addition, the combinational therapeutic method has a combinational system of topical formulations and light irradiating devices for intra-mucosal and transvaginal drug delivery. The intravaginal light therapy as disclosed and described in the foregoing detailed description further facilitates the following advantages over the existing prior art:
Dilation flexibility and compliance of e atrophied vagina with the device structure and size and shape;
light irradiation based intra-vaginal delivery of therapeutic substances into the vaginal membrane without causing damage to the vaginal membrane or underlying tissues;
photokinetic intra-vaginal delivery of therapeutic substances into the vaginal membrane without denaturation and/or degradation of the therapeutic substance(s) being administered;
application of pulsed visible, near-infrared and mid-infrared light wavelengths onto vaginal tissues as a therapy to stimulate collagen production thereby thickening the vaginal walls and consequently rejuvenating vaginal tissues;
administering the compositions containing hyaluronic acid into vaginal tissue to retain the moisture and to relieve symptoms of vaginal dryness;
incorporating tissue-humidifying and tissue conditioning substances into therapeutic compositions to be applied to the vaginal tissue;
incorporating compounds with a biological activity of collagen production stimulation into the therapeutic formulation to be applied to the vaginal tissue;
enabling the use of the therapeutic formulation as a personal lubricate to facilitate enhanced comfort during sexual intercourse;
use of the therapeutic formulation comprising compounds with antibacterial, antifungal, and antiviral properties as a treatment to prevent the diseases of the vagina;
provision of vaginally insertable light devices of optimal size, shape, and structure to cause the dilation of constricted atrophied vaginal openings;
provision of vaginally insertable light devices of optimal size, shape, and structure to cause the enlargement and enhanced the flexibility of atrophied and confined vaginal spaces in order to provide enhanced comfort during sexual intercourse;
reduction in vaginal rigidity and stiffness thereby promoting vaginal tissues and structures to become more compliant and flexible allowing for greater comfort during sexual intercourse; and,
insertion of the vaginally insertable light device advantageously dilating the rigid vaginal space to make the tissues more compliant and more flexible allowing for enhanced comfort during sexual intercourse.

Additional embodiment of the present invention provides the advantages of the vaginally insertable light device having a vibrational mode of operation.

A further embodiment of the present invention provides the advantage of vaginal pelvic floor exercise and corresponding tracking system.

Although this invention has been disclosed in the context of certain illustrative embodiments and examples, it will be understood by those skilled in the art that the present invention extends beyond the specifically disclosed embodiments to other alternative embodiments and/or uses of the invention and obvious modifications and equivalents thereof. Accordingly, it is intended that the scope of the present invention herein disclosed should not be limited by the particular disclosed embodiments described herein.

### 3. Photokinetic Device and Method of Use

In order to ameliorate and overcome one or more drawbacks and disadvantages associated with the prior art and to provide additional advantages, a device and method for photokinetic intravaginal trans-membrane delivery of a biologically active composition is provided and illustrated herein in the form of a non-limiting and exemplary embodiment. Additional features and advantages are realized through the techniques of the disclosure of the present invention. Other embodiments and aspects of the disclosure of the present invention are described in detail herein and are considered a part of the claimed invention.

Firstly, the compositions, methods, and devices described herein preferably use pulsed incoherent light to facilitate enhanced tissue permeation, tissue deposition and delivery of topically applied biologically active therapeutic formulations into the outer most surface of the non-keratinized vaginal tissues and to facilitate tissue permeation and deposition within the interstitial space of the vaginal mucosa layer structures.

In accordance with further aspects of the present disclosure, a vaginally inserted therapeutic light device comprising of electronic circuits producing pulsed electronic signals driving LEDs to produce pulsed light of a selected light wavelength, selected pulse rate, and selected pulse duration is disclosed. The electronic circuitry, LEDs and battery power supply is housed in a spherical clear plastic, clear silicone, clear hardened glass or other clear material that allows the light to reflect within the housing without substantial light intensity losses (total internal reflection) until the light is extracted from the light device by scattering or tissue contact that effectively extracts the light from within the light device out and onto the biologically active substances on vaginal tissue surface.

In some embodiments, compositions containing only biologically active substances are used as delivery media, whereas in other embodiments, biologically active substances are used in combination with other components as a delivery media carrier solvent for the biologically active substance.

Methods and devices employing pulsed incoherent light are used to actively transport a biologically active therapeutic medium containing hyaluronic acid from the outer surface of the vagina or vaginal mucosal membrane into the interstitial space of the tissues. This provides many advantages, including binding or attaching the hyaluronic acid to the vaginal mucosa allowing the vaginal mucosa to retain moisture and water thereby hydrating the tissue. Furthermore, the pulsed incoherent light transports other components of the biologically active therapeutic topical composition containing vitamins and collagen production stimulating substances into the vaginal mucosa. This also advantageously affects the tissue by stimulating the vaginal tissue to produce collagen and elastin and thereby increase the tissue thickness, flexibility and compliance as a means of treating and reversing vaginal atrophy.

Methods and devices employing pulsed incoherent light are used to actively transport a biologically active compound into the outer surface of the vagina and mucous membrane. The biologically active compound, in the invention, is being comprised in a pharmaceutically acceptable composition that is suitable and convenient for application onto the vaginal tissue surfaces and is supportive of the vaginal flora. The biologically active compound on the tissue surface is then available for translocation from the tissue surface into the vaginal tissue, this translocation facilitated by cyclic incoherent visible and infrared light irradiation. This provides many advantages, including the ability to create a pathway for enhanced drug delivery without causing damage to the vaginal tissue membrane while being able to reversibly excite biologically active molecules with light energy without degrading or denaturing them chemically. In addition, the rate of delivery of the biologically active component can be controlled, sustained, or substantially stopped, by modulating the wavelength, pulse rate, duty cycle and intensity of the light being used to photokinetically actuate, propagate and translocate the biologically active compound from the non-keratinized tissue surface into the underlying vaginal mucosa and vaginal genital tissues.

Additionally, the use of a vaginally inserted light emitting device containing at least one, but more preferably more than one light source, permits light to expose a biologically active medium over a wide surface area of the vagina. Therapeutic biologically active substances deposition concentrations into the vaginal mucosa can be enhanced through the use of compositions, methods and devices described herein.

Furthermore, light other than light intended to facilitate photokinetic delivery, i.e. therapeutic light that directly and advantageously affects tissues of the vagina can be used concurrently or in sequenced activation being incorporated into the vaginally inserted light device and in addition to the selected photokinetic light within the same light emitting device. Herein, at least one therapeutic light emitting source, particularly generating light in the red (600 nm-700 nm) and near-infrared (700 nm to 1400 nm) wavelength range can also be used. The light emitting sources are controlled by different electronic pathways and circuitry and powered by the integral light device battery pack. In the preferred embodiment, all light sources, regardless of the intended effect, are pulsed with the ON time start of the pulse being in phase with each other while the OFF time may vary for one light source to the others within the same vaginally inserted device. In the preferred embodiment, there is an OFF phase where there is no light, or the light is being substantially attenuated.

The electronic control package controller can differentially generate pulse rate and pulse duration signals to the several light sources within the vaginally inserted light device. Wherein, for example, all the light sources within the device are driven at a selected pulse rate of, for example, 1000 cycles per second while the photokinetic light source is differentially driven with a pulse duration (or duty cycle) of, for example 1%, the near-infrared light source is driven at, for example, 10% duty cycle and the red light source is driven at, for example, a 50% duty cycle. This differentiation of light pulse control allows for the concurrent use of selected photokinetic light with therapeutic light. Pulsing the light allows a cooling/quenching period secondarily allowing the tissue to dissipate heat from absorbed light energy. Another aspect of the invention, vaginal tissue damage denaturation by heating the tissues above 42° C. is avoided by light pulsing and duty cycle attenuation. Non-thermal light irradiation is a preferred embodiment of the invention.

Described herein is a novel platform technology pertaining to enhanced and facilitated permeation kinetics of compounds into and through a tissue by the application of selected pulsed incoherent light (photokinetic method). This photokinetic facilitated tissue deposition technology allows for administration and delivery of a wide range of compounds having a wide range of molecular weights into intact vaginal mucosal tissues, without damage to the tissues or chemical changes to the molecule being delivered while substantially increasing the concentration of the therapeutic compound within the tissue matrix as a means of enhanced biologically active substance tissue deposition.

Therefore, one salient feature of the methods and systems of the invention is its superior safety feature, which can be particularly valuable for treating atrophic vaginitis, vaginal atrophy and vaginal dryness. For example, since the penetration distance is a function of the time exposed to the light source (e.g., LED), the ability to regulate the flux rate of the biologically active substance, including the ability to target the delivery of the substance at a specific depth within the vaginal mucosa, enables targeted delivery of certain biologically active substances into vaginal tissues (without causing damages to the vaginal or leading to unpredictable effects resulting from non-targeted systemic absorption of the substance).

Another safety feature of the subject delivery method resides in the fact that the delivery method does not damage human vaginal tissues, and the active ingredients being delivered are not transformed or photochemically altered into potentially harmful substances by the applied light energy. Additionally, red light (600-700 nm) and near-infrared light (700-1400 nm) wavelengths selected do not kill or directly harm the beneficial resident bacterial flora within the vagina. In the preferred embodiment, light energy is not used to denature a biologically active substance or initiate a photochemical reaction. Conversely, in photodynamic therapy, other selected light is positioned to irradiate photosensitizing compounds to chemically change the compound by light energy to produce toxic substances. In the preferred embodiment, light energy does not photochemically change the vaginally applied biologically active substance or produce toxic by-products.

Pulsing the light is advantageous to reduce or eliminate the possibility of creating harmful exogenous heat from light irradiation of tissues. It is well understood that certain chromophores and interstitial water residing in or around tissues absorb selected light wavelengths. This light absorption is converted to heat which may cause an amount of heat that could denature the tissues of the cellular components that absorb the light irradiation. By pulsing the light and providing a period wherein a reduced amount of light is absorbed by the tissues or tissue cellular components, any heat generated is allowed to dissipate thereby preventing tissue damage from heat. Pulsing may be accomplished by modulating the ON time (duty cycle) of the applied light or reducing the total light energy output of the illuminating source. Therefore, selected light pulsing by providing discrete periods of ON/OFF illumination is advantageous while providing light energy to the tissues and at the same time removing the possibility of damaging heat secondary to the thermal response of absorbed light energy.

Accordingly, in one respect, the invention provides a method for enhanced photokinetic intra-membrane delivery of a biologically active substance having a molecular weights of 165 Da to 2.2 kDa to the vaginal mucosa of a subject, the method comprising: (1) applying said biologically active composition to a cellular vaginal mucosa surface of the subject; (2) inserting the selected light device into the vagina; (3) illuminating said formulation on said cellular surface of the vagina with a pulsed incoherent light having a selected wavelength, pulse rate and duty cycle; and, (4) allowing said biologically active substance in said formulation to permeate said vaginal mucosal membrane surface and deposit within the vaginal intestinal space in concentrations higher than can be achieved by passive permeation thereby effecting photokinetic vaginal interstitial delivery of the biologically active substance.

Accordingly, in another respect, the invention provides a method for enhanced photokinetic intra-membrane delivery of a biologically active substance having a molecular weights of about 165 Da to about 2.2 kDa onto the vaginal mucosa of a subject; the method comprising: (1) applying said biologically active composition to a cellular vaginal mucosa surface and vaginal genital tissues of the subject; (2) inserting the selected light device into the vagina; (3) illuminating said formulation on said cellular surface of the vagina with a pulsed incoherent light having a selected wavelength, pulse rate and duty cycle; and, (4) allowing said biologically active substance in said formulation to permeate said vaginal mucosal membrane surface into the interstitial space of the tissue, thereby effecting photokinetic intra-mucosal delivery of the biologically active substance.

In certain embodiments, the pulsed incoherent light is selected from the group consisting of fluorescent, ultraviolet, visible, near-infrared, mid-infrared, LED (light emitting diode), laser diodes, organic electroluminescent material (OLED) and halogen light. In certain embodiments, the selected light has a wavelength range from about 260 nm to about 1,400 nm.

In certain embodiments light wavelengths of LEDs are specified as for example 660 nm however it is understood the polychromatic light output of an LED has a peak range and a wider range wavelength range at the 50% output. It is understood the true light generated by an LED, or organic electroluminescent material (OLED) for example, includes a range of light wavelengths in addition to the peak emission wavelength for an LED or OLED, for example, may be 660 nm however the emission wavelength range may be 660 nm±12 nm. Laser diodes are also polychromatic and therefore not true lasers, however, have a narrower range of light emission for example 650 nm±4 nm. Therefore, light wavelength specifications and descriptions encompass normal polychromatic of the LED, OLED or laser diode or other light sources.

In certain embodiments, the selected light has a wavelength range from about 340 nm to about 1400 nm. In the preferred embodiment LED (light emitting diode) or OLED (organic electroluminescent material) or laser diode light sources are used.

In certain embodiments, the visible light has a wavelength range from about 340 nm to about 800 nm.

In certain embodiments, the near-infrared light (NIR) has a wavelength range from about 700 nm to about 1,400 nm.

In certain embodiments, the mid-infrared light has a wavelength range from about 1300 nm to about 4,000 nm.

In certain embodiments, the pulse rate is between about 1.7 cycles per second (cps) and about 2000 cps, or about 1.7 cps and about 8000 cps.

In certain embodiments, the pulse rate is between about 24-4000 cps.

In certain embodiments, the ON time duty cycle is between about 10% and about 75%.

In other embodiments, a discrete ON time and a discreet OFF time, in the range of, for example, 1% ON/99% OFF to 99% ON/1% OFF (including any integer values in between, such as 2% ON/98% OFF, 5% ON/95% OFF, 10% ON/90% OFF, 15% ON/85% OFF, etc.) are all contemplated embodiments of the invention. In certain embodiments, the method further comprises adjusting the intramucosal membrane flux rate of the biologically active substance by modulating the light energy ON and OFF time periods.

In other embodiments, two separate light wavelengths are combined and be pulsed and modulated in phase with each other. For example, a red 660 nm LED is pulsed at 1000 cycles per second with a 50% ON time while a NIR LED 940 nm is pulsed at 1000 cycles per second with a 15% ON time with both light sources ON time starting at the same time while the OFF time for each is selected. Therefore, the ON cycle starting times for both light sources are in phase with each other.

In other embodiments, three separate light wavelengths can be combined and be pulsed and modulated in phase with each other. For example, a red 660 nm LED is pulsed at 1000 cycles per second with a 50% ON time combined with red 830 nm LED is pulsed at 1000 cycles per second with a 50% ON time while a NIR LED 940 nm is pulsed at 1000 cycles per second with a 15% ON time with all three light sources ON time cycle starting at the same time while the OFF time for each is selected. Therefore, the ON cycle starting times for the three light sources are in phase with each other. If more than three light sources are used than it is understood that the ON cycle start time of the pulse cycle for all are in phase and the OFF time may differ for each, however there is a discrete time when all the light sources are OFF or significantly attenuated.

In the preferred embodiment, when LED light sources are used, a three LED diode surface-mount LED package, commonly known as an SMD, is used within the device. The SMD LED diodes emitting peak wavelengths of about 660 nm±20 nm, 830 nm±20 nm and 940 nm±50 nm. In the preferred embodiment, the three-wavelength LED SMD package provides a compact and efficient use of space within the intravaginal device as well as providing a more uniform distribution of the individual light wavelengths directed onto the light irradiated tissues.

In certain embodiments the pulsed light duty cycle (the discrete ON time and the discreet OFF time) is modulated to limit the absorbed light energy secondarily eliminating heat buildup from light energy absorption of specific light wavelengths. For example, in the preferred embodiment, tissue temperatures of greater than 42° C. is avoided by duty cycle modulation. More preferably, the higher limit of tissue temperatures is less than or below 42° C. Preferably, tissue thermal heating by applied light energy is avoided specifically to eliminate vaginal tissue damage by heat while still allowing the light-based actuated drug delivery effect as well as the increased vaginal blood flow effects from slightly elevated tissue temperatures.

In certain embodiments, the method further comprises adjusting the permeation flux rate of the biologically active substance by modulating the light energy, in order to deliver all or substantially all biologically active substance intramucosally (within the mucosa).

Another aspect of the invention provides a device for photokinetic intra-membrane, intramucosal or intra-vaginal delivery of a biologically active substance to a subject, said device comprising: (1) a generator that provides an oscillating electrical pulse; (2) at least one LED (light emitting diode), and/or OLED (organic electroluminescent material) and/or laser diode that receives the oscillating electrical pulse and responds by providing a pulsed incoherent light emission; and, (3) a therapeutic composition applied to the vaginal surfaces positioned between the vaginal surfaces and the light emitting device wherein the therapeutic composition is positioned to receive the light energy.

Another aspect of the invention provides a device to provide therapeutic light onto the vaginal tissues for said device comprising: (1) a generator that provides an oscillating electrical pulse; (2) at least one LED (light emitting diode), and/or OLED (organic electroluminescent material) and/or laser diode that receives the oscillating electrical pulse and responds by providing a pulsed incoherent light emission; and, (3) allowing the light to illuminate the vaginal surfaces positioned to receive the therapeutic light energy from the vaginally inserted light device.

In certain embodiments, the pulse generator is a repeat cycle square electrical wave pulse generator.

In certain embodiments, the device further comprises a spherical or cylindrical plastic, glass or other clear material that houses the light emitting source, wherein at least one light emitting diode or other light source is embedded and housed within the spherical housing.

In certain embodiments, the device further comprises a tapered plastic, glass or other clear material that houses the light emitting source, wherein at least one light emitting diode is embedded and housed within the tapered housing. In this embodiment the tapered housing provides a progressive increase in diameter cross-section to provide a progressive dilating feature to stretch and enlarge a constricted vaginal opening and/or progressively stretch an atrophied and constricted vagina as a method to treat vaginal stenosis (a narrowed and contracted introitus opening of the vagina) and vaginismus (vaginal muscle spasm).

In certain embodiments, the vaginally inserted light device is comprised of substantially optically clear material with encapsulated and potted light sources optically coupled to the device housing using substantially optically clear material for the encapsulation and potting of the light source without an air gap between the light source emitting surface and the housing of the light device body.

In certain embodiments, the vaginally inserted light device is comprised of an optically clear material with encapsulated light source layers of electroluminescent materials (OLED) and/or combination of OLED material and LEDs and electronic drivers.

In certain embodiments, the optically clear material is polymethyl methacrylate, polycarbonate, glass, plastics, epoxies or silicone rubber and/or combinations therefore.

In certain embodiments the tapered housing provides a progressive increase in diameter cross-section to provide a progressive dilating feature to stretch and enlarge a constricted vaginal opening introitus and/or progressively stretch a constricted atrophied vagina as a method to treat vaginal narrowing secondary to vaginal conditions such as vaginal stenosis or vaginismus.

In certain embodiments the tapered housing provides a progressive increase in diameter cross-section to provide a progressive dilating feature to stretch and enlarge a constricted anal opening and/or progressively stretch a constricted rectum as a method to treat anal or rectal narrowing secondary to anal conditions such as post hemorrhoidectomy stenosis.

Other specific embodiments of this aspect of the invention are described above and not repeated verbatim here.

In yet another aspect, the invention provides a method for enhanced vaginal blood flow: (1) applying a formulation comprising the biologically active substance to a cellular vaginal mucosa surface of the subject; (2) inserting the light device into the vagina; (3) illuminating said formulation on said cellular surface of the vagina with a pulsed incoherent light having a selected wavelength, pulse rate and duty cycle; and, (4) allowing said light to be absorbed by the vaginal tissue and trigger physical and/or metabolic pathways that then causes an increase in blood flow within the tissues as a method to increase sexual arousal and/or decrease pain from sexual penetration.

In certain embodiments, the subject receiving treatment is a female human.

In certain embodiments, the vaginal mucosa is non-porated intact vaginal tissue, or the mucosa is porated by chemical, electrical, and/or physical means.

In certain embodiments, the cellular surface is the internal surfaces of the vagina and mucus membrane.

It should be understood that any embodiments of the invention described herein, including those described under different aspects of the invention, can be combined with one or more other embodiments of the invention.

Furthermore, using the subject therapeutic formulation allows an additional layer of safety, since the subject intra-vaginally applied composition is produced from safe ingredients, many of which are natural or synthesized to be substantially similar to natural substances, and do not contain potentially harmful substances to the vagina, to the beneficial resident bacterial flora or to the body of the treatment recipient. The combination of the subject intra-mucosal delivery technology with the subject therapeutic composition enhances delivery of the active ingredient, but also provides the above safety features valued in vaginal drug delivery for treatment of atrophic vaginitis, vaginal atrophy, and vaginal dryness.

The non-keratinized surface of the vaginal mucosa is permeable to many drug compounds. Drug flux permeation is generally inversely proportional to the molecular weight of the compound; as the molecular weight increases the permeation flux decreases. Generally, permeation is concentration gradient dependent in the absence of applied active transport energy. The photokinetic actuated translocation system allows for increased permeation into the surface of the membrane and increased biologically active substance deposition into the interstitial space of the membrane by applied selected light energy this process providing a higher biologically active substance deposition within the tissue in greater achievable concentrations compared to passive concentration gradient permeation conditions in the absence of applied light energy. In context, within the embodiment of the invention, the applied selected light energy produces higher biologically active substance concentrations within the vaginal tissues compared to achievable biologically active substance concentrations by passive permeation concentration gradient conditions for tissue permeation.

In accordance with further aspects of the present disclosure, a therapeutic biologically active substance composition comprising aqueous mixtures of lubricants, moisturizers, film producing agents, peptide mixtures, tissue conditioning agents and antioxidants including, but not limited to: 0.1-5% sodium hyaluronate (a sodium salt of hyaluronic acid); peptide mixtures (0.1% to 5%), tissue conditioning agents (0.1% to 5%), plant derived extracts (0.1% to 5%), solvents, co-solvents, solubilization enhancing cyclodextrins, mucoadhesives (substances that adhere to the surface of mucus membranes) (0.1% to 5%); gelling agents, drugs (0.001%-10%), chemicals and preservatives (0.01% to 3%) are disclosed.

In some embodiments the therapeutic composition, or parts thereof, function as a mucoadhesive that attached to the mucus membrane of the vagina.

In some embodiments the therapeutic composition is a lubricant that eases the vaginal insertion of the therapeutic light device into the vagina.

In some embodiments the lubricating therapeutic composition is an optical coupling agent with a refractive index higher than water; water having a refractive index of about 1.333. The optically coupling biologically active composition couples the surface of the light emitting device with the vaginal tissues herby increasing the light transmission from the device into the optically coupled tissues.

Additionally, in accordance with further aspects of the present disclosure, a therapeutic composition comprising an aqueous mixtures comprising lubricants, moisturizers, mucoadhesives, film producing agents, peptide mixtures, tissue conditioning agents, antioxidants being one or more of the biologically active substances including but not limited to: 0.1-5% sodium hyaluronate, Tremella fuciformis sporocarp extract 0.1-5%, human stem cell growth media 0.1-5%, Saccharomyces ferment 0.1-10%, phytosteryl canola glycerides 0.1-2%, ubiquinone 0.1-2%, caprylic/capric triglyceride 0.1-10%, Resveratrol-cyclodextrin molecular complex 0.1-1.0% 1,3 propanediol 0.1-5%.

Numerous biologically active substances useful in the skin care field/industry for skin conditioning can also be delivered into the vaginal tissue to rejuvenate and condition the vaginal tissue using the subject photokinetic intradermal delivery methods, optionally with the subject topical formulations. One category of tissue conditioning biologically active substance includes all skin and mucosal components (such as collagen, natural or its synthetic forms) that could be readily replaced, replenished, or supplemented with exogenous molecules delivered by the subject methods. Merely to illustrate (and accordingly not limiting), the following categories of skin and mucosal beneficial molecules may be delivered using the subject methods: antioxidant photoprotection molecules, such as Vitamin E (tocopherols, in particular Alpha-tocopherol, and Tocotrienols), ascorbate, carotenoids (Beta-carotenoid-/Vitamin A, lycopene, zeaxantine, lutein), Coenzyme Q (Ubiquinone, Idebenone), glutathione (including glutathion derivates: ethyl ester, cystine, etc.), Alpha-glycolic acid, SOD (Superoxide Dismutase), catalase, glutathione peroxydase, reductase, taurine, and Alpha-lipoic acid; polyphenols such as epsilon-viniferin, mixtures of resveratrol, quercetin, fisetin, ferulic acid, and epsilon-viniferin, and mixtures of polyphenolic, in particular stilbene and/or falvonol, oligomers and/or polymers; molecules with healing power, such as melanin, glycerol, peptides, or growth factors; glycoproteins, such as sialic acid (moisturizing action, infective prevention), GAGs (chondroitin sulphate, dermatan sulphate, cheratan sulphate, eparin, eparan sulphate, hyaluronates), decorin; collagene fibers—other fibers, such as hydroxyproline (+Fe, +Vitamin C), hydroxylysine, glycine, tropocollagen, reticulin, keratin, elastin, MMPs (matrix metalloproteinases); beta-glucans; phytosterols; Anti-aging molecules, such as arginine, citrulline, ceramides, carnosine, lysine, inositol, cysteine, squalene, squalane, chitin, sericine, peptides, or growth factors.

In certain embodiments, the vaginally applied formulation comprises a solution and a solvent. In certain embodiments, the solvent is an aqueous or an organic solvent.

In certain embodiments, the aqueous solvent is an aqueous solution of water or water with co-solvents, or an aqueous solution containing other water miscible solvents. In certain embodiments, the formulation comprises an emollient system wherein one or more components is an oil or oil-like compound. In certain embodiments, lipohilic compound constituents are molecularly included within cyclodextrins or cyclodextrin derivatives to produce substantially water-soluble compositions or parts of the composition.

In certain embodiments, the water is the sole solvent in the aqueous solution without a co-solvent or emulsion system. Certain naturally occurring lipophilic compounds such as plant derived polyphenols, flavonoids, stilbenes and sub-categories thereof (e.g. essential oils, resveratrol, quercetin, fisetin, kaempferol, ferulic acid) are anticipated to be constituents in the vaginally applied composition. Those skilled in the art, recognize these plant polyphenols, essential oils and other plant derived compounds are beneficial to tissues though multiple pathways while also possessing anti-inflammatory, antioxidant, antibacterial, antifungal and antiviral properties.

Cyclodextrin (and cyclodextrin derivatives) molecular inclusion of these lipophilic natural compounds provide substantial water solubility while leaving the included compound chemically unchanged and without the use of harmful or pharmaceutically unacceptable solvents, such as for example ethanol and/or dimethyl sulfoxide (DMSO). Oil-water emulsion systems based on surfactants and/or caprylic/capric triglyceride and/or other similar co-solvent systems, significantly partition lipophilic substance away from the water phase of the composition diminishing the inherent anti-bacterial and antifungal properties necessitating the incorporation of exogenous antimicrobials such as phenoxyethanol or parabens for formulation stability. For example and without limitation, essential oils, resveratrol, quercetin, fisetin, ferulic acid can be molecularly included within 2-Hydroxylpropyl-β-cyclodextrin allowing the poorly water-soluble compounds to become substantial water-soluble without the use of surfactants, co-solvents or oil carriers. See: Marques HMC. A review on cyclodextrin encapsulation of essential oils and volatiles. Flavour and Fragrance Journal, 25:5(2010), 313-326; Bertacche V, et al. Host-Guest Interaction Study of Resveratrol With Natural and Modified Cyclodextrins. Journal of Inclusion Phenomena and Macrocyclic Chemistry 55:3 (2006) 279-287; Borghetti G S. et al. (2009). Quercetin/β-Cyclodextrin Solid Complexes Prepared in Aqueous Solution Followed by Spray-drying or by Physical Mixture. AAPS PharmSciTech, 10:1 (2009), 235-242; Guzzo M R, et al. Study of the Complexation of Fisetin with Cyclodextrins. The Journal of Physical Chemistry A, 110:36 (2006) 10545-10551; Jullian, C, et al. Cyclodextrins-Kaempferol Inclusion Complexes: Spectroscopic and Reactivity Studies. Journal of Solution Chemistry, 40:4 (2011) 727-739; Wang J, et al. Characterisation of inclusion complex of trans-ferulic acid and hydroxypropyl-β-cyclodextrin. Food chemistry, 124:3 (2011) 1069-1075.

The inherent antimicrobial, antifungal and antiviral properties of the polyphenols is maintained in the non-partitioned aqueous carrier. Cyclodextrin molecular inclusion has also be shown to increase the bioavailability of poorly-soluble Chemical Class II compounds such as plant polyphenols. See Brewster M E & Loftsson T. Cyclodextrins as pharmaceutical solubilizers. Advanced drug delivery reviews 59:7 (2007) 645-666. Those skilled in the art, would recognize the advantages of molecular inclusion formulation constituents for an aqueous based vaginally applied topical therapy eliminating the need for surfactants, cosolvents and exogenous synthetic formulation stability antibacterials.

In certain embodiments, the formulation comprises lipophilic biologically active substances included within the cyclodextrin to form a complex that is applied to the vaginal tissue cellular surface and irradiated with pulsed infrared light (700-1400 nm). Without being bound by theory, the pulsed infrared light irradiation is cyclically absorbed and dissipated by oxygen-hydrogen bounds of the cyclodextrin molecule there from causing the lipophilic compounds included within the cyclodextrin to be released from the cyclodextrin/lipophilic compound complex and deposit onto the cellular surface of the vaginal tissue. The pulsed infrared light irradiation of the cyclodextrin/biologically active substance complex residing on the cellular surface being a method of enhanced and accelerated release of the lipophilic biologically active substance from the cyclodextrin complex in amounts greater compared to passive dynamic equilibrium release without infrared light irradiation. The irradiation of vaginal topically applied biologically active ingredient cyclodextrin complexes, with pulsed infrared light emitted from an intravaginal light device directed onto the topically applied complexes, initiates a release of the ingredient from the cyclodextrin complex in dissociation rates higher than dynamic equilibrium dissociation rates without infrared irradiation. The pulsed infrared light irradiation being a method of enhanced biologically active substance release from a cyclodextrin complex there from as a method of light-facilitated intra-tis sue deposition of a biologically active substance.

In certain embodiments, the topical formulation has a higher refractive index than water (water having a refractive index of about 1.333) and when applied to the vaginal tissues, the topical formulation provides an optical coupling between the vaginally inserted light emitting surface of the light device to the vaginal tissues in contact with the light device separated by the topical formulation. The optical coupling of the light device with the tissues mediated by the topical formulation between the light device and the tissue, reduces tissue surface light back-scattering and thus affects an increase in forward light transmission from the light device into the bulk of the vaginal tissue.

In certain embodiments, the formulation comprises cyclodextrin included lipophilic compounds in combination with water soluble proteins, peptides, antioxidants, drugs, humidicants (e.g. hyaluronic acid, methylcellulose, carbomer, thiolated poly(acrylic acid), poloxamer, celluloseacetophthalate, hydroxy ethyl cellulose, poly(amidoamine) dendrimers, poly(dimethyl siloxane) and poly(vinyl pyrrolidone) and drugs.

In certain embodiments, the formulation comprises a gelling agent and/or a mucoadhesive.

In certain embodiments, the biologically active substance comprises a chemical, an antioxidant, a peptide, an antibody, a protein, a plant extract, or a mixture thereof.

In certain embodiments, the biologically active substance comprises a drug.

In certain embodiments, the drug is a vitamin or tissue conditioning compound.

In certain embodiments, the biologically active substance comprises or further comprises chemicals and said chemicals comprise a polar or a non-polar compound.

In certain embodiments, the biologically active substance is a tissue conditioning agent, or a therapeutic agent directed to improve the structure of the vaginal tissue.

In certain embodiments, the biologically active substance comprises or further comprises: peptides, a protein selected from the group consisting of enzymes, non-enzymes, antibodies, and glycoproteins.

In another embodiment, the biologically active substance can be emulsified. For example, lipophilic compounds, such as vitamins A, D, and E or other plant-derived glycerides, can be dispersed in an aqueous solvent with an emulsifying agent, such as surfactant or self-emulsifying oils can be added.

In another embodiment, the biologically active substance can be encapsulated within natural or cyclodextrin derivatives, liposome, micelle, nanoparticle and/or other encapsulating technology such as Qusome® technology wherein the active ingredients reside is a phase separate from the carrier phase. Active ingredients may be in an encapsulated form for suspension within the composition. Nanoparticles, and compounds included in cyclodextrin derivative molecules are used to enhance water solubility, enhance compound stability and improve tissue permeation and compound bioavailability.

Likewise, in the absence or presence of a solvent, the biologically active agent according to the invention can also be combined with a carrier or adjuvant, a substance that, when added to a therapeutic, speeds or improves its action. Examples of adjuvants include, for example, Freud's adjuvant, ion exchanges, alumina, aluminum stearate, lecithin, buffer substances, such as phosphates, glycine, sorbic acid, potassium sorbate, partial glyceride mixtures of saturated vegetable fatty acids, glycerin, waters, salts or electrolytes, such as potassium sulfate, disodium hydrogen phosphate, sodium chloride, zinc salts, colloidal silica, magnesium, trisilicate, cellulose-based substances and polyethylene glycol. Adjuvants for gel-based forms may include, for example, hyaluronic acid, carbomers, carboxymethylcellulose, sodium carboxymethylcelluslose, polyacrylates, polyoxyethylene-polyoxypropylene-block copolymers, polyethylene glycol, and wood wax alcohols.

Although not required to facilitate transmembrane delivery, membrane-penetrating agents, for example, propylene glycol, DMSO, oleic acid, laurocapram (Azone), cineol, liposomes, nanosomes, and cyclodextrin included compounds can also be present in the compositions according to the invention.

One embodiment of the invention relates to compositions for photokinetic intradermal, interstitial, intramucosal and intramembrane delivery of a biologically active substance using preferably pulsed incoherent light. The composition may comprise at least one biologically active substance as the delivery medium.

The composition may alternatively comprise a biologically active substance and a solvent. The percentage of a biologically active substance in a solvent can be in the range of between 0.0001 to 99.9999% (w/v). Preferably, the biologically active substance is present in a concentration range of between about 0.01% to about 4% (w/v). More preferably, the biologically active substance is present in a concentration range of between about 0.1 mg/ml to about 40 mg/ml in the solvent or, alternatively, between about 0.01% to about 4% (w/v, w/w). Due to the high level of permeation achieved by the methods and devices described herein, low concentrations of the biologically active substance in a solvent or in other compositions described herein can be used for efficient intramembrane delivery.

The composition may instead comprise a biologically active substance, a gelling agent and a solvent. The percent concentration of the gelling agent in a solution comprising the biologically active substance can vary depending on the type of gelling agent used. For example, Klucel is typically used at 1% (w/v), Natrasol at 1.5% (w/v), Carbopol at 0.75% (w/v), and hyaluronic acid is used in a concentration range of about 0.25% to 5% (w/v) depending on molecular weight of the hyaluronic acid. For example, high molecular weight hyaluronic acid may be used at a lower concentration than low molecular weight hyaluronic acid as there is an increase in gelling capacity with higher molecular weight hyaluronic acid.

Still further, the composition may comprise a biologically active substance, an antioxidant, and a solvent. Preferably, an antioxidant is included in the composition.

Compositions according to the invention may comprise a biologically active substance, a gelling agent, an antioxidant and a solvent. The gelling agent preferably is present in the composition at a concentration of between 0.1% and 10% (w/v).

The biologically active substance of the above compositions may be selected from the group consisting of chemicals, active compounds, drugs, antibiotics, peptides, hormones, proteins, DNA, RNA, plant extracts of the various active ingredients, and mixtures thereof.

The drug may be selected from the group consisting of analgesics, anesthetics, antianxiety drugs, antibacterials, antibiotics, anticonvulsants, antidepressants, antiemetics, antifungals, antihistamines, antihypertensives, anti-inflammatories, antineoplastic, antipsychotics, antipyretics, antivirals, barbiturates, beta-blockers, bronchodilators, corticosteroids, cytotoxics, diuretics, expectorants, hormones, hypoglycemics, immunosuppressives, muscle relaxants, sedatives, sex hormones, sleeping drugs, tranquilizers, vitamins, moisturizers, tissue conditioning agents and lubricants.

The compositions according to the invention may also comprise antibiotics as the biologically active substance. Antibiotics according to the invention are selected from the group consisting of amoxycillin, ampicillin, penicillin, clavulanic acid, aztreonam, imipenem, streptomycin, gentamicin, vancomycin, clindamycin, ephalothin, erythromycin, polymyxin, bacitracin, amphotericin, nystatin, rifampicin, teracycline, coxycycline, chloramphenicol, and zithromycin. In one embodiment, the antibiotic is amphotericin B.

Similarly, in another embodiment of the invention, the biologically active substance is a peptide selected from the group consisting of glycine-tyrosine (Gly-Tyr), valine-tyrosine-valine (Val-Tyr-Val), tyrosine-glycine-glycine-phenylalanine-methionine (Tyr-Gly-Gly-Phe-Met) (SEQ ID NO: 1), tyrosine-glycine-glycine-phenylalanine-leucine (Tyr-Gly-Gly-PheLeu) (SEQ ID NO: 2), and aspartic acid-arginine-valine-tyrosine-isoleucine-histidine-proline-phenylalanine (Asp-Arg-Val-TYr-Ile-His-Pro-Phe) (SEQ ID NO: 3). Human stem cell growth media is an example of a peptide mixture use herein.

The hormone may be selected from the group consisting of methionine enkephalin acetate, leucine enkephalin, angiotensin II acetate, β-estradiol, methyl testosterone, progesterone, and insulin.

A protein may be selected from the group consisting of enzymes, non-enzymes, antibodies, and glycoproteins. In one embodiment of the invention, the protein is an enzyme.

Compositions according to the invention can also contain a gelling agent in combination with the biologically active agent and solvent. The gelling agent may be selected from the group consisting of hydroxyethyl cellulose, hyaluronic acid, Natrasol®, pectines, agar, alginic acid and its salts, guar gum, pectin, polyvinyl alcohol, polyethylene oxide, cellulose and its derivatives, propylene carbonate, polyethylene glycol, hexylene glycol sodium carboxymethylcellulose, polyacrylates, polyoxyethylene-polyoxypropylene block copolymers, pluronics, wood wax alcohols, and tyloxapol. In one embodiment, the gelling agent is hydroxypropyl cellulose. In one embodiment, the gelling agent is hyaluronic acid and or its derivative sodium hyaluronate.

The composition may also comprise a solvent that is an aqueous or organic solvent. In one embodiment, the aqueous solvent is water. In yet another embodiment, the aqueous solvent is an aqueous solution of ethyl lactate or propylene glycol. The water may be purified by means such as reverse osmosis, filtration and/or distillation.

The composition applied to the vaginal tissue, the vaginal delivery medium or delivery medium according to the invention is comprised of a biologically active substance itself or any mixture of a biologically active substance with a solvent, a gelling agent, an antioxidant or combinations of antioxidants and polyphenols, a carrier or adjuvant, a membrane-penetrating agent, emulsifier, one or more different biologically active substances, polymers, excipients, coatings and/or combinations thereof. In essence, the biologically active substance or substances can be combined with any combination of pharmaceutically acceptable components to be delivered to the cellular surface of the vagina and vaginal genital tissues by the method described herein, e.g., photokinetic transmembrane delivery. The biologically active substance does not have to be dissolved in a solvent but can be suspended in a solid form or encapsulated or molecularly included within a nanoparticle or liposome or emulsified in a solvent. The composition applied to the vaginal tissue, or delivery medium can take the form of an aqueous or an organic liquid, a cream, a paste, a powder, patch or other drug eluding membranes. The vaginally applied solution can also comprise biologically active substances encapsulated in microspheres, nanospheres, and/or cyclodextrin included molecules of in combinations with dissolved and/or suspended chemicals in liquid or semi-solid form.

The invention described herein is particularly useful for light energy facilitated intra-membrane delivery of compounds containing chromophores into non-keratinized tissues containing also containing chromophores by the application of pulsed light with a distinct ON and OFF phase or a period of attenuated light illumination. Without being bound by any particular theory, it is believed that the molecular chromophores of the topical carrier, the drug and/or the tissue absorbs photon energy and/or the energy from excited constituents in the topically applied composition during the ON light phase. As the chromophore returns to ground state during the OFF pulse phase, it generates kinetic energy. With each pulse of incoherent light, the chromophore's vibration will incrementally clear a pathway through the non-keratinized vaginal mucosa into the interstitial space of the vaginal tissue.

Also consistent with the theory and not being bound by any theory, specific chemical bonds stretch, twist and change confirmation in response to light irradiation. Oxygen-hydrogen bonds in water, in the water of the delivery medium, in water within the tissues, in compounds that have oxygen-hydrogen bonds within their chemical structure and oxygen-hydrogen bonds on the surface of cyclodextrin derivative compounds, absorb discrete wavelengths of infrared light and respond by stretching the distance between the oxygen and hydrogen molecule in response to the light irradiation excitation. The oxygen-hydrogen bond reverts to the normal resting state in the absence of light irradiation. Vibrational energy is produced during the dissipation of energy from the excited state to the resting state. Cyclic, pulsed irradiation of discrete wavelengths of infrared light causes a net vibration within the water of the delivery medium, in water within and on the surface of the tissues, in biologically active substances and within the oxygen-hydrogen bonds on the surface of cyclodextrin derivative compounds. The said light irradiation then causing a net vibration and kinetic force clearing a path for photokinetic delivery and tissue deposition of topically applied biologically active substances, as well as substances that are molecularly included within the cyclodextrin molecule host carrier, into the tissues having received the topically allied biologically compounds and being irradiated by and receiving the selected cyclic, pulsed irradiation of selected infrared light discrete light wavelengths.

Still further consistent with this theory, chemical and heat energy can cause molecular shape changes and increases in Brownian motion within a system. These energies cannot be easily cycled and are not directional. Light energy, on the other hand, is readily defined by wavelength and can be easily controlled for cycle time/cycle duration stimulation and incident direction. Molecular conformational changes as a result of optical stimulation by the absorption of light energy is widely known phenomenon exhibited by many classes of drugs and tissue molecular constituents with degrees of shape change determined by the individual molecular structure and the wavelength of the incident light. The conformational changes initiated by the exemplary photokinetic method are reversible; the molecules tend to revert to a resting state as the optical energy is dissipated during the OFF part of the cycle. Accordingly, cyclic light stimulation creates a repeated and reversible molecule shape change or gross physical movement or translocation from one place to another on a molecular scale.

In addition, and again not being bound by theory, according to Le Chatelier's principle, if a system in chemical equilibrium is subjected to a disturbance, it tends to change in a way that opposes this disturbance. Molecular systems move in the direction to reduce the external stimuli; molecules tend to move away from an energy source. Kausar et al. have published a paper detailing how a molecule can be translocated around a surface as a response to light stimulation, generally by moving away from the light energy. See Kausar et al. Photo controlled translational motion of a microscale solid object on azobenzene-doped liquid-crystalline films. Angew Chem Int Ed. 48 (2009) 2144-2147. In the photokinetic light-based drug delivery system, the drug is applied to the tissue surface and illuminated from a direction opposite the tissue. If the molecule is to escape the light energy stimulation, then it moves in a direction away from the stimulation and into the tissue. The repeated cycling of the molecular shape confirmation adds a gross physical movement. The possible interaction of cycled incident incoherent light on the membrane itself may also cycle the tissue structure and transmembrane pathways. These cycled membrane changes may impart a pumping action on the pathways through the tissue with cycled structure sizes between tissue connective fibers further allowing enhanced tissue permeation.

In addition to compositions, the invention also provides methods of photokinetic delivery of biologically active substances using pulsed incoherent light. One method includes applying a solution comprising a biologically active substance in a solvent onto a cellular surface, illuminating the solution and the solvent on the non-keratinized cellular surface of the vagina with a pulsed incoherent light having a selected wavelength, selected pulse rate and selected duty cycle and allowing the solution to permeate the cellular surface of the vagina into the underlying interstitial space depositing the biologically active substances in higher concentrations than if allowed to permeate without the application of the selected pulsed light.

In another embodiment, the method includes applying a solution comprising a biologically active substance, a solvent and a gelling agent onto the cellular surface of the vaginal genital area, illuminating the solution on the cellular surface with a pulsed incoherent infrared light having a selected wavelength, selected pulse rate and selected duty cycle and allowing the solution to permeate the cellular surface and deposit into the underlying tissue structures.

In yet another embodiment, the method includes applying a solution comprising a biologically active substance, a solvent, a gelling agent and an antioxidant agent onto a cellular surface; illuminating the solution on the cellular surface with a pulsed incoherent light having a selected visible and infrared light wavelength, selected pulse rate and selected duty cycle; and allowing the solution to permeate the cellular surface. In a preferred embodiment, the cellular surface is an outer layer of a non-keratinized mucus membrane of the vagina of a mammal.

In yet another embodiment, the method includes applying a solution comprising a biologically active substance, a solvent, a gelling agent, on to a vaginal surface, illuminating the solution on the cellular surface with a pulsed incoherent light having a selected wavelength between 600 nm and 1400 nm, with a selected pulse rate and selected duty cycle and causing a rejuvenating effect on the vaginal tissue. In a preferred embodiment, the cellular surface is an outer layer of a vaginal mucus membrane of a mammal with the light emitting device is positioned within the vagina allowing light to illuminate the solution applied to the vaginal membrane.

In yet another embodiment, the method includes applying a solution comprising a biologically active substance, a solvent, a gelling agent, onto a vaginal surface, illuminating the solution on the cellular surface with a pulsed incoherent light from more than one light sources having a selected light wavelength between 400 nm and 4000 nm, with a selected pulse rate and selected duty cycle and alternating and cycling through a program of between at least two light wavelengths between 400 nm and 4000 nm, having selected pulse rates and duty cycles and causing a rejuvenative effect on the vaginal tissue. In a preferred embodiment, the cellular surface is an outer layer of a vaginal mucus membrane of a mammal with the light emitting device is positioned within the vagina allowing light to illuminate the solution applied to the vaginal membrane.

Other objects, aspects, features and goals of the present invention are better understood from the following detailed descriptions of representative (non-limiting) embodiments.

### 4. Illustrative Embodiments of Intravaginal Light Devices

Human exterior surface skin and human vaginal mucosa are similar in structure except that human exterior skin has a heavily keratinized layer (stratum corneum). This keratinized layer is the primary barrier for drug permeation into keratinized exterior skin surfaces. The vaginal mucosa is absent a keratinized tissue surface layer and is thus more permeable than human skin in general, however, the mucosa remains a barrier for drug permeation into the interstitial space within the vaginal tissue under the surface of the vaginal mucosa. The invention provides methods, compositions and devices that enhance the vaginal permeation of topically applied biologically active substances into the interstitial space below the tissue surface in tissues of the vagina. Methods described herein generally reflect the enhanced permeation into vaginal tissue membrane and more importantly the deposition of drugs and biologically active substances into the interstitial space of the vaginal tissue membrane thereby making the biologically active substances available to the cells within the vaginal membrane for a biological effect.

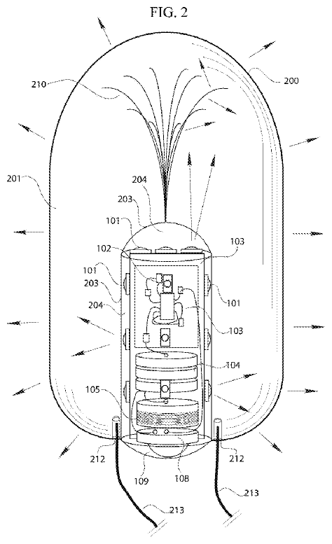

Now, with reference to vaginal light devices as disclosed in FIG. 2 through FIG. 12, the illustrative configurations and applications are not meant to restrict the embodiments of the various contemplated devices in practice. Several attributes described within the individual illustrations can be mixed to form a separate device, all of which are anticipated by the invention. In all the illustrative embodiments, the vaginal light devices comprise at least one LED and/or one layer of OLED material and/or one laser diode, an electronic drive circuit and an electrical power source; the device is intended to be partially or fully inserted into the vagina of a human. In one embodiment, a biologically active composition is administered onto the vaginal tissues and/or on to the vaginal light device to aid in the insertion of the device by lubrication. This vaginally applied biologically active composition further provides a therapeutic function within the vaginal tissues in combination with the vaginally inserted light device.

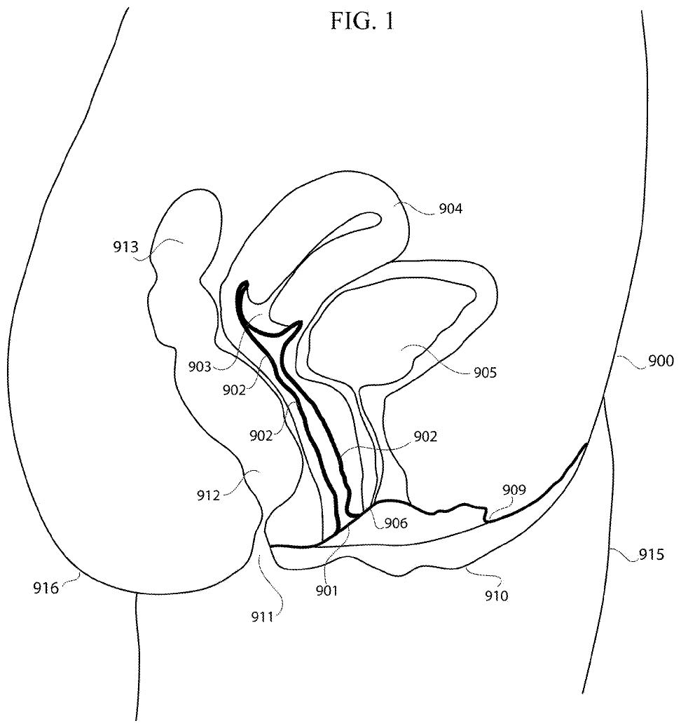

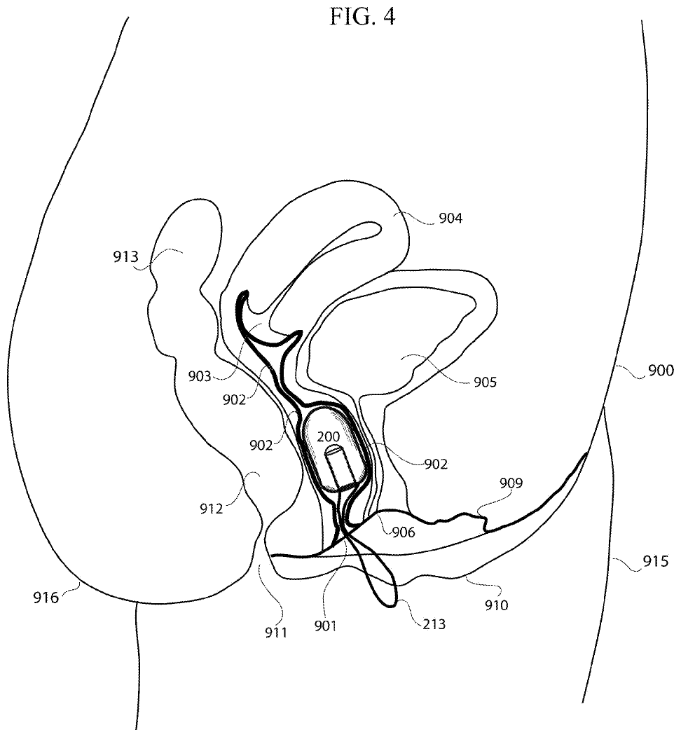

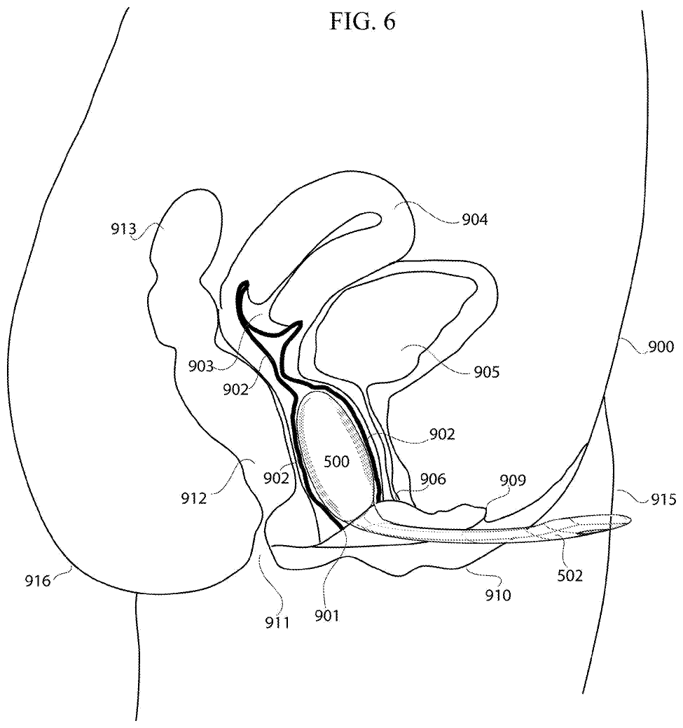

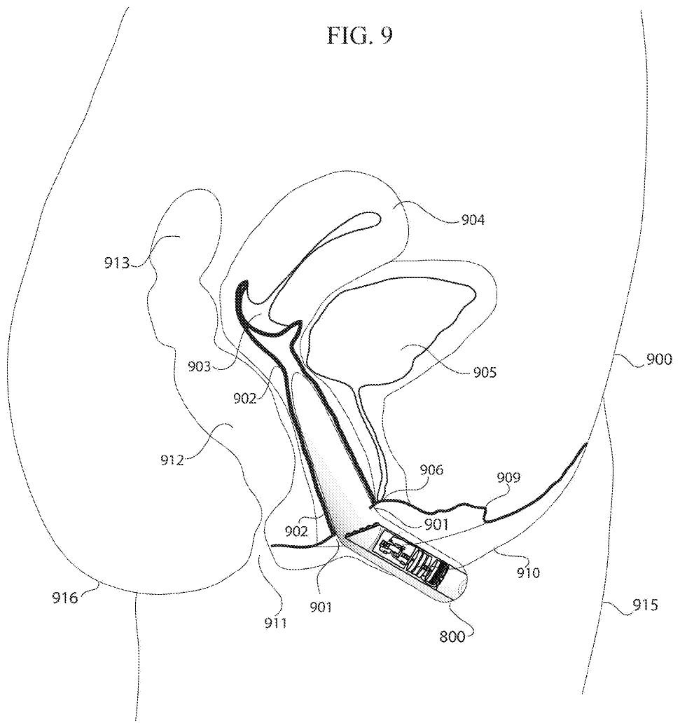

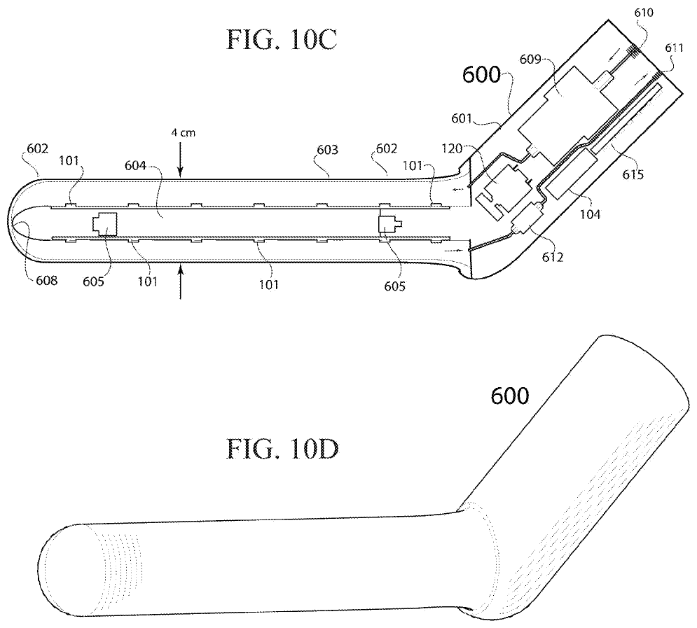

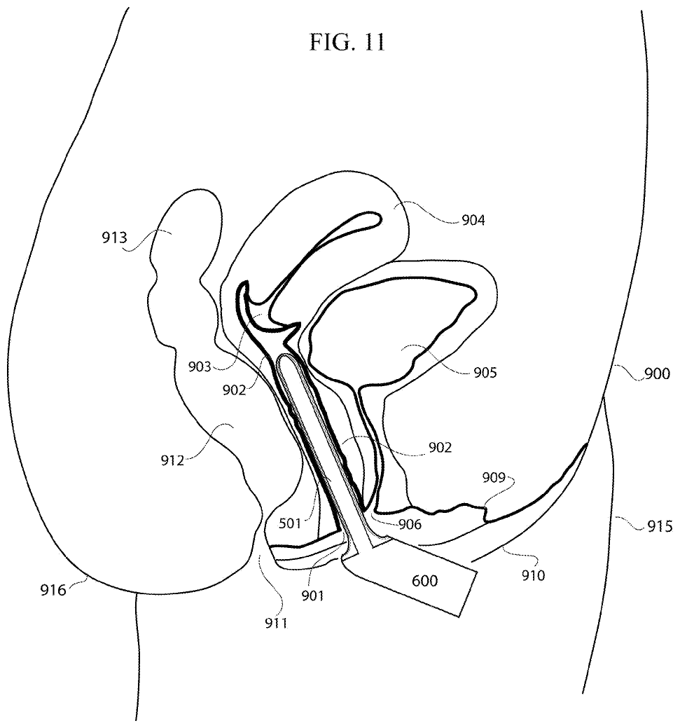

In order to illustrate how the intra-vaginal light devices will be applied or inserted into the female anatomy FIG. 1 represents a diagrammatic view of the female lower abdomen cross section showing the atrophied vagina 902 and surrounding anatomical structures. This illustration is used for continued reference of the placement of the intravaginal devices as shown in FIG. 4, FIG. 6, FIG. 9, FIG. 11 and FIG. 12.

Female anatomy (FIG. 1) illustrates as follows: The female reproductive organ, including the vaginal opening 901, the vaginal canal 902 the cervix 903 and the uterus 904, is positioned in the lower abdomen 900 anterior to the buttock 916, rectum 912, distal rectum 913, anus 911 and posterior to the urinary bladder 905 that terminates into the urethra 906 all structures within the lower abdomen 900. The exterior rectum opening 911 from the rectum 912 and is posterior to the vaginal opening (introitus of the vagina) 901 of the vagina 902. The distal vagina 902 terminates at the cervix 903 of the uterus 904. Immediately adjacent to the vagina is the urethral opening 906 leading from the urinary bladder 905. The exterior vaginal genitalia labia and labia majora 910 is the exterior genitalia structure including the clitoris 909.

Referring now to FIG. 2, a representative illustration of a fully insertable intravaginal light device. The intravaginal light device housing 200 is comprised of an optically clear smooth material 201 such as clear medical grade silicone, polymethyl methacrylate, polycarbonate, polyethylene terephthalate, hardened glass, crystal glass, quartz or any other optically clear material that can be fabricated into the size and shape desired. The size of the device may be variable to accommodate the differences in vagina size and vaginal opening. The device may be larger than the intended vaginal target site to allow for some vaginal stretching as part of intended use. The size of the intravaginal device in the preferred embodiment would allow for placement within the vagina without causing pain while large enough to remain in place without ejection from its own weight by gravity alone.

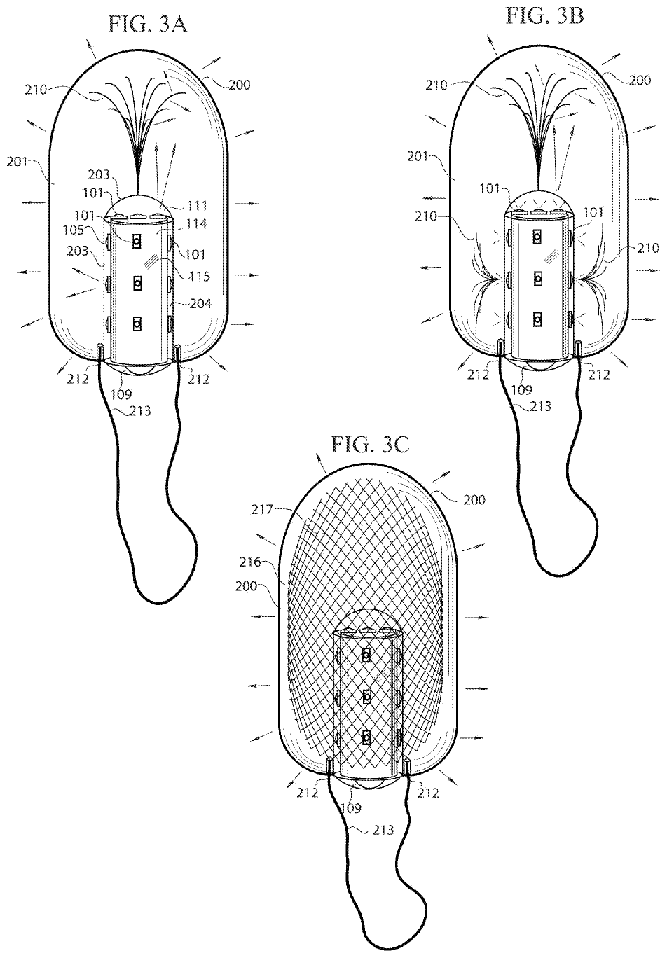

With continued reference to FIG. 2, the housing is manufactured with a cavity 203 that is intended to accommodate the electronics package 103. The electronics package 103 is comprised of an electronic pulse generator 102 that provide pulsed electronic drive current to the various LEDs 105. In this view the electronic package components 103 are shown, in later views (FIG. 3A, FIG. 3B and FIG. 3C) the preferred embodiment shows an electronic package housed within a cylindrical housing along with LEDs 101 mounted on the outside surface of the electronics package housing coated with a reflective surface directing the light away for the electronic package housing. The circuitry allows for separate control of at least one group of LEDs at a selected wavelength. The electronic package may contain three or more separate groups of LEDs at various light wavelengths. The electronic package also contains batteries 104 to power the circuitry and LEDs. In the preferred embodiment, the batteries can be re-charged with a magnetic induction system 105 with integral charging control circuitry. The electronic driver circuitry is controlled by a mechanical or magnetically operated switch or radio frequency controlled switch 108. The switch mechanism 108 is sealed with a flexible cover 109. The entire electronics control package is imbedded, encapsulated and potted within the intravaginal device with optically clear epoxy or other suitable electronic embedding substance 204.

The device housing 200 composition 201 is selected to be substantially free of optical absorption in the light wavelengths used therein. The light emitted from LEDs 101 will radiate from the device 200 or will be totally internally reflected within the device until the light is extracted by contact with the vaginal tissue or the light incident angle provides extraction from within the device. The device housing 200 may have mechanically etched, laser etched, or cast in place structures 210 that are intended to scatter and redirect the light without causing light loss while directing the light out of the device.

The housing 200 also contained attachment points 212 so that a flexible retrieval “string” loop 213 can be affixed to the housing structure. The flexible retrieval string 213 is comprised of a flexible monofilament material or a coated multi-strand string structure. The flexible retrieval string 213 is intended to be flexible and comfortable when the housing is positioned within the vagina. The string would also be useful to aid in vaginal insertion and removal of the device. The string 213 may also serve as an antenna for external communication to a device controlling program application. The surface of the string material 213 is intended to have a smooth surface free of pits or defects. The entire apparatus 200 exteriors including the retrieval string 213 and the switch covering 109 are to be smooth and free of surface defects, pits or crevasses that could harbor bacteria. The device is intended to be easily washed and sanitized with water.

FIG. 3A illustrates the LEDs 101 mounted on the surface of a cylindrical housing 115 for the electronics package contained inside. In the preferred embodiment shows the cylindrical electronic package 115 contains all of the electronics, batteries and charging circuitry. The cylindrical housing 115 is coated with a reflective surface painted polishing mirror or another reflective surface 115, directing the light away for the electronic package housing; the LEDs 101 are mounted on top of the reflective surface with light output directed away from the electronics package 115. The entire electronics package 115 is embedded with an optically clear material 201 such as optically clear epoxy or similar material within the optically clear intravaginal device housing 200. The flexible retrieval string 213 is shown in its entirety in this view. In the preferred embodiment, the flexible retrieval string 213 is a closed loop.

Referring now to FIG. 3B, the device housing 200 may have multiple mechanically etched, laser etched, or cast in place structures 210 that are intended to scatter and redirect the light without causing light loss while directing, reflecting or scattering the light out of the device away from the LED 101 light sources is illustrated.

Referring now to FIG. 3C another method to diffuse and scatter the light by mechanically etching, laser etching, or casting in place light diffusing structures 217 within the intravaginal device housing is illustrated. In this embodiment an inner structure 216 is first fabricated and etched 217 then a second layer of optically clear material 201 is placed over the etched surface creating the intravaginal light device 200 with a smooth and substantially surface defect free exterior that will not harbor bacteria and allow for easy insertion within the vagina and easy cleaning after use. The light diffusing etching pattern 217 is a singular example of a light diffusing method. Those skilled in the art understand that many light diffusing patterns could be incorporated and would be useful.

Referring now to FIG. 4; in this view, the intravaginal light device 200 is inserted into the vagina 902 beyond the vaginal opening 901 (introitus of the vagina). The retrieval string 213 is used to help position during insertion and for removal after treatment. The retrieval string 213 is fabricated from material that is flexible and soft to allow for comfort while also having a closed waterproof surface structure that exclude surface voids or defects that may harbor bacteria. The retrieval string assembly 213 may also have an integral antenna that is exterior to the body cavity allowing for wireless communication with external electronic controller and monitoring devices such as a Smartphone application or independent wireless controller monitor.

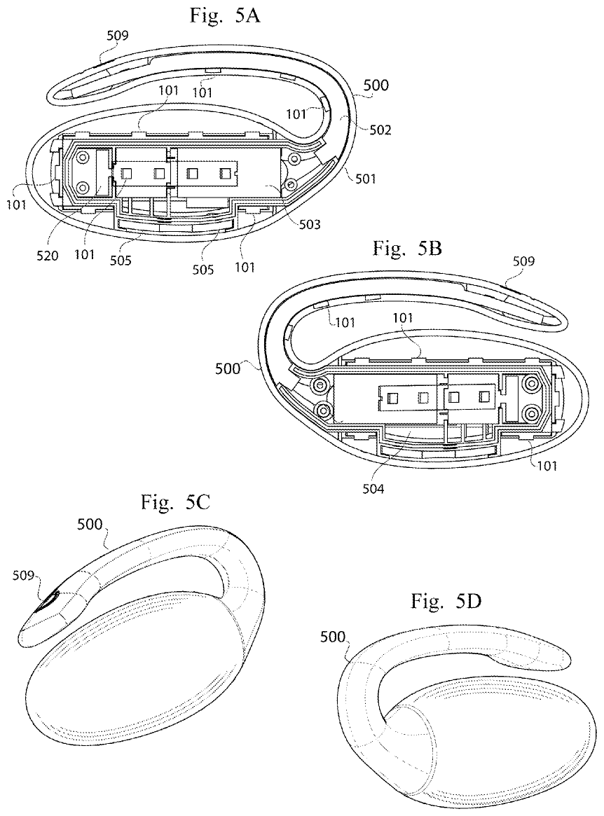

FIG. 5A representative sectional illustration of a fully insertable intravaginal light device and a reflected view as shown in FIG. 5B in a flexible transparent housing with a flexible retrieval stem. The flexible and pliable intravaginal device provides superior comfort. The device has a multitude of LEDs 101, LED driving circuitry 503, vibration generating motor 520, battery 504 and battery charging induction coil 505 all housed in a waterproof housing 501. The retrieval stem 502 is configured to aid in the vaginal placement so that the light device body and stem is in close proximity to the urethra opening to provide direct light irradiation and vibration to the urethral opening to treat urinary incontinence. The retrieval stem 502 has LEDs 101 positioned to irradiate the external genitalia to initiate increased blood flow to the tissues for enhance sexual wellness. The distal retrieval stem 502 has a progressive pressure switch 509 to operate the functions for example LEDs ON, vibration ON, OFF. An antenna can be added to the stem 502 for remote control and information transfer to an external device. Additional functions such as vaginal pressure sensors to track vaginal exercise and vaginal contraction can be added within the flexible housing.

FIG. 5B is a reverse reflected view of FIG. 5A additionally showing the internal battery 504, LEDs 101 and the progressive pressure switch 509.

FIG. 5C illustrates a perspective surface view of the external surface of a fully insertable waterproof flexible, nonrigid, clear silicone intra-vaginal device shown in FIG. 5A.

FIG. 5D illustrates a reverse perspective surface view of the external surface of a fully insertable waterproof flexible, nonrigid, clear silicone intra-vaginal device FIG. 5B.

FIG. 6 illustrates how the flexible fully insertable intravaginal light device 500 is poisoned within the vagina to provide internal vaginal light irradiation as well as external genitalia light irradiation from the stem 502.

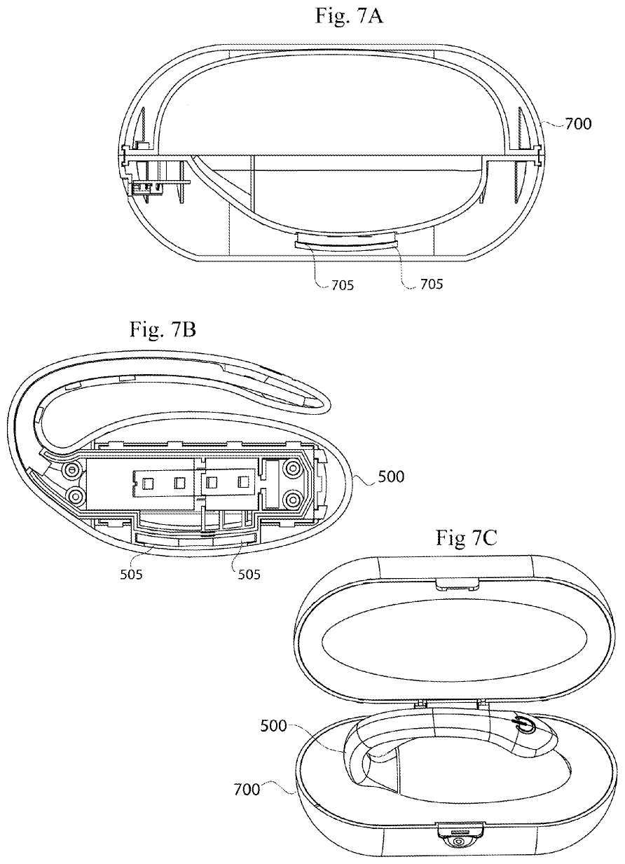

Now turning to FIG. 7A, FIG. 7B and FIG. 7C illustrating the induction charging system FIG. 7A in relationship to the intravaginal unit as shown in FIGS. 5A and 5B. FIG. 7A illustrates a cross sectional view of the charging housing 700 with an integral electrical induction coil 705 positioned within the charging housing 700. FIG. 7B shows the position of the intravaginal unit induction charging coil 505. FIG. 7B shows the position of the induction coil within the flexible intra vaginal device previously shown and oriented as shown in FIG. 5B. FIG. 7C illustrates an perspective surface view of the intravaginal unit 500 positioned within the induction charging housing 700.

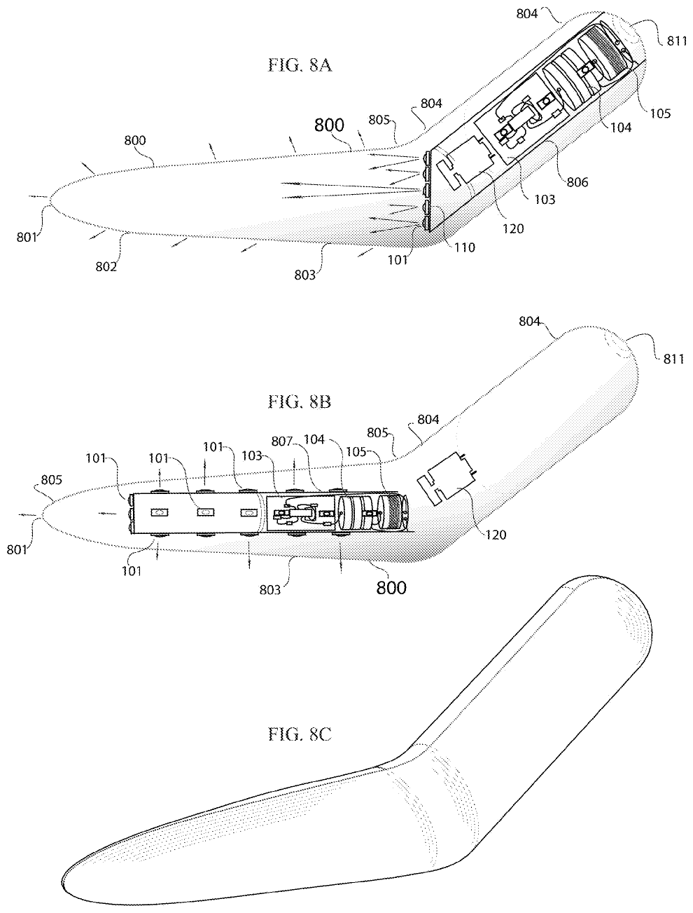

Now turning to FIG. 8A and FIG. 8B illustrating two configurations of a rigid or semi-rigid vaginal light device 800 intended to provide progressive vaginal opening dilation and internal vaginal dilation of an atrophied vagina. The vaginal light device 800 has an increased diameter from the distal end 801 to the proximal area 805 of the vaginal insertion shaft area 803. The device is cylindrical in shape with a tapered vaginal introduction end 801 with progressively increasing diameter along the shaft 803. The handle area 804 is angled away from the shaft area 805 to allow ergonomic ease of use.

In the configuration of FIG. 8A, an electronics package 103 is positioned in the handle area 804. The electronics package consists of an LED array 110 assembly of LEDs 101 receiving a pulsed electrical driving current from the control circuitry 103. Internal batteries 104 supply the drive current to the control circuitry 103 and are induction charged by the induction coil assembly 105. The unit is switched ON by a waterproof switching sensor 811 that also acts with an ON timer function. The LED array is encapsulated, potted and optically coupled with the light emitting shaft area 803 within the device 800. Light generated by the LED array 110 progresses within the substantially transparent insertion area 803. When the transparent assembly comes in contact with vaginal tissue, light is extracted from within the device into the vaginal tissue. The entire unit 800 is waterproof and free of surface irregularities, crevices or seams that could harbor bacteria. The light emitting area 803 is substantially transparent to visible and infrared light. The unit also has the additional option of a vibration feature to treat urinary incontinence generated by a vibration generator 120.

Now turning to, FIG. 8B shows an alternate configuration wherein the electronic package consisting of LEDs 101 electronic drive circuitry 103 battery 104 and induction charging system 105 are positioned within the area intended to be inserted into the vagina 803. The electronic package and its components are encapsulated, potted and optically coupled with the light emitting shaft area 803 within the device 800. The unit also has the additional option of a vibration feature to treat urinary incontinence generated by a vibration generator 120.

The dilating device as shown in FIG. 8A and FIG. 8B would also have an additional utility for anal dilation to treat anal or rectal narrowing or anal stenosis secondary to rectal surgeries (i.e. hemorrhoidectomy) or other conditions that may constrict or narrow the rectal canal wherein anal dilation is desired.

FIG. 8C is a perspective surface view of FIG. 8A and FIG. 8B illustrating the progressively tapered feature for vaginal dilation.

Referring now to FIG. 9 as a representative view of the female anatomy having placement of the representative of vaginal light devices 800, described in FIGS. 8A and 8B.

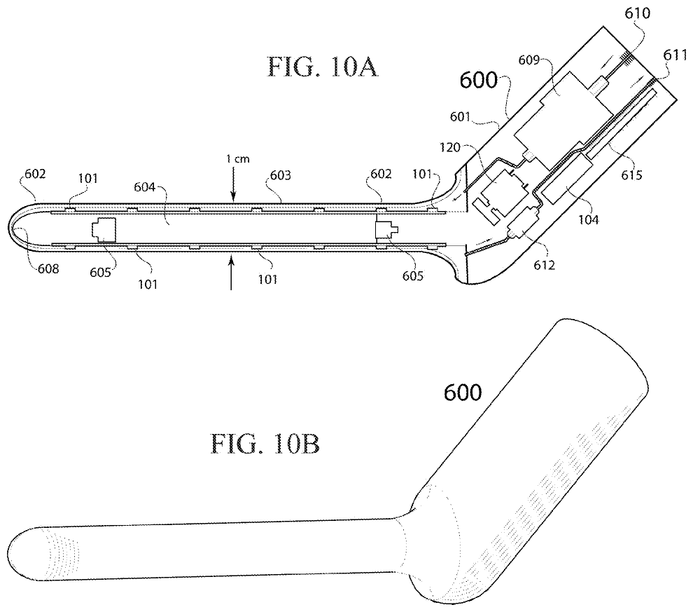

Now turning to FIGS. 10A and 10C that illustrates a flexible vaginal opening and vaginal canal pulsatile dilating device 600 in the relaxed or deflated position as shown in FIG. 10A and the expanded, inflated position as shown in FIG. 10C. The device 600 has a handle area 601 and an insertion area 602. The insertion area has a semi-rigid core 604 that houses LEDs 101 and proximity sensors 605. The insertion area is covered by a flexible transparent or semi-transparent sheath 603 that is attached 608 to the distal end of the semi-rigid core 604 and the handle area 601. The handle area 601 houses a micro-air pump that fills and pressurizes the flexible sheath 603. Electronic proximity sensors 605 and sheath pressure relief valve sensor 612 are coupled with the unit electronic drivers and logic processing circuitry 615 to process pressure and volume measurements in order to derive a tissue compliance function. Pressure sensor 612 provides the total pressure within the flexible sheath 603. Proximity sensors 605 are arranged on the semirigid insertion area 602 internal surface 604 to provide a dimensional feedback of the flexible sheath 603. The pressure sensor/air relief valve 612 modulates the sheath pressure and reports sheath pressure values to the electronic logic circuitry 615. Electronic logic controller and LED driver circuitry 615 is powered by battery 104. Air intake 1-way valve 610 allows air to enter the micro-air pump. Air relief valve 611 and air intake valve 610 are spring loaded normally-closed so that water cannot enter the device if submerged for cleaning after use. A vibration emitting motor 120 is positioned to provide a vibration effect onto the urethral opening to treat urinary incontinence. FIG. 10B is a perspective surface view of the of the deflated device 600.

FIG. 10D is a perspective surface view of the of the inflated and expanded device 600.

FIG. 11 and FIG. 12 illustrate the flexible vaginal opening and vaginal canal dilating device within the female vagina and further illustrates the dilation effect from the relaxed or deflated position as shown in FIG. 10A and the expanded, inflated position as shown in FIG. 10D.

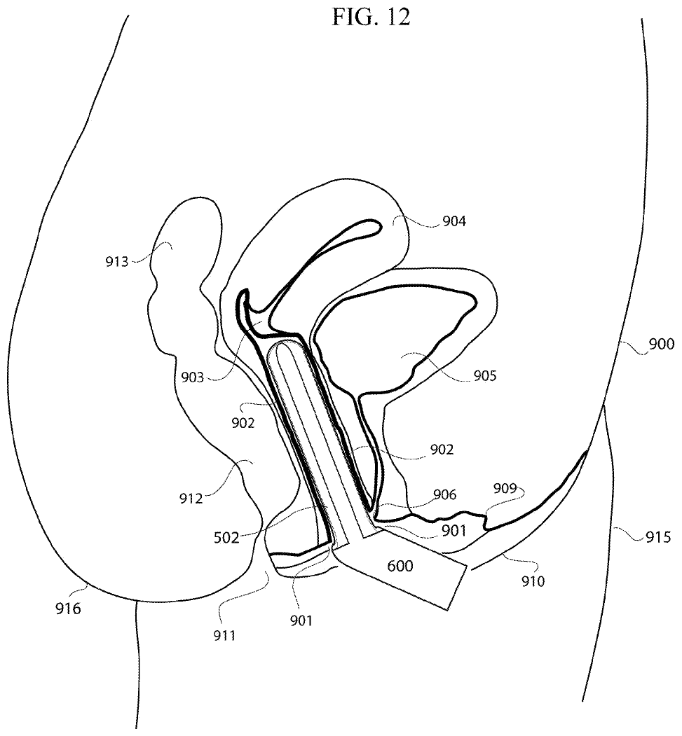

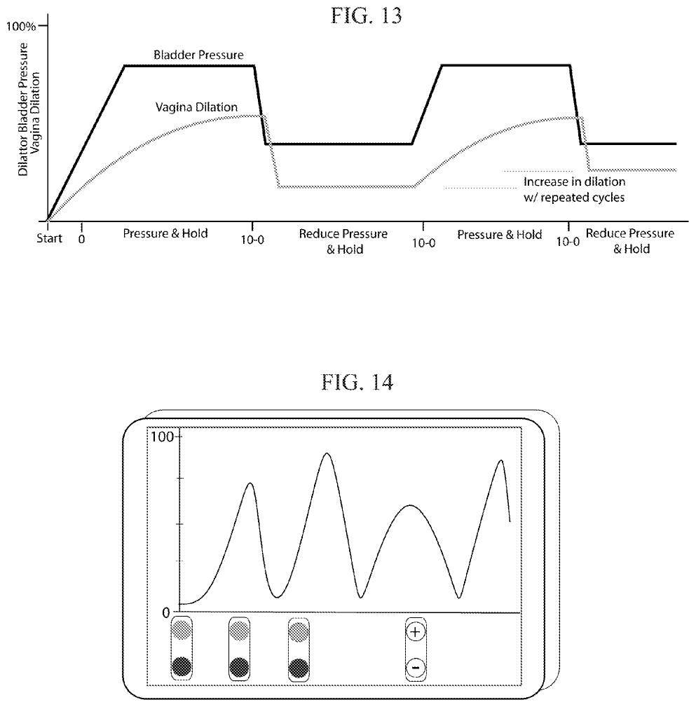

FIG. 13 is a visual representation illustrating the relationship between internal vaginal flexible sheath pressure and vaginal dilation achievable with the specified sheath pressure. The amount of pressure exerting within the flexible sheath of the device and the amount of dilation achieved by the pressure exerted derives a tissue flexibility or tissue compliance computational value that is use to track therapeutic efficacy and user feedback. A pulsatile sheath pressure function is shown.

FIG. 13 illustrates one iteration of a remote controller and informational tracker for the pulsatile dilating device. The controller tracker may be configured to control basic device functions such as LED light ON/OFF, vibration control ON/OFF, dilation pressure settings and pressure limits, relaxed sheath pressure limit and tissue compliance computational normalization factor setting. Additional features such as pelvic floor exercise monitoring by incorporation of a pressure strain measurement sensor within the insertable device along with associated electronic communication circuitry are anticipated in the invention. Mechanical vibration initiation features are anticipated in any of the illustrative embodiment of the invention to accommodate exercises and treatments for urinary stress incontinence. Various secondary control and monitoring features, for example but not limited to, vaginal dilation, tissue compliance measurements, urethral opening vibration, of the device configurations can be combined or omitted. The underlying device elements of producing pulsed light irradiation directed toward vaginally applied biologically active compositions and light irradiation of vaginal tissues are consistent and unifying with all the illustrated devices and their operation shown or obvious to those skilled in the art.

### EXAMPLES

### Human Testing of Light Devices and Vaginally Applied Topical Compositions

Several vaginally applied biologically active formulations were tested on a human subject with concurrent pulsed light irradiation within the vagina with composition examples below:

### Example 1

Example 1

| Ingredient | INCI Name | % of formulation |
|---|---|---|
| Water | Aqua | 92.95 |
| 1,3-propanediol | Propylene glycol | 5.0 |
| (plant derived) | | |
| Hyaluronic acid | Sodium hyaluronate | 1.25 |
| Vita C ® | Aminopropyl ascorbyl phosphate | 0.2 |
| Vitamin B5 | panthenol | 0.2 |
| Vitamin B3 | nicotinamide | 0.2 |
| L-Carnitine | carnitine | 0.2 |

### Example 2

Example 2

| Ingredient | INCI Name | % of formulation |
|---|---|---|
| Water | Aqua | 90.85 |
| Hyaluronic acid | sodium hyaluronate | 4.0 |
| 1,2-propanediol | propylene glycol | 3.0 |
| (plant derived) | | |
| Salt | sodium Chloride | 0.45 |
| Vita C ® | aminopropyl ascorbyl phosphate | 0.2 |
| Vitamin B5 | panthenol | 0.5 |
| Vitamin B3 | nicotinamide | 0.5 |
| L-Carnitine | carnitine | 0.5 |

### Example 3

Example 3

| Ingredient | INCI Name | % of formulation |
|---|---|---|
| Water | Aqua | 85.4 |
| Hyaluronic acid | sodium hyaluronate | 4.0 |
| Mushroom extract | tremella fuciformis sporocarp extract | 4.0 |
| Growth media | human stem cell growth media | 4.0 |
| Fungi ferment extract | saccharomyces ferment | 0.5 |
| Canola oil extract | phytosteryl canola glycerides | 0.5 |
| Enzyme CoQ10 | ubiquinone | 0.5 |
| Coconut oil extract | caprylic/capric triglyceride | 0.5 |
| 1,3-propanediol | propylene glycol | 0.5 |
| Other ingredients | | 0.1 |

### Example 4

Example 4

| Ingredient | INCI Name | % of formulation |
|---|---|---|
| Water | aqua | 84.9 |
| Hyaluronic acid | sodium hyaluronate | 4.0 |
| Mushroom extract | tremella fuciformis sporocarp extract | 4.0 |
| Growth media | human stem cell growth media | 4.0 |
| Fungi ferment extract | saccharomyces ferment | 0.5 |
| Trans-resveratrol | resveratrol | 0.5 |
| cyclodextrin complex | | |
| 2-HP-β-cyclodextrin | cyclodextrin | 1.0 |
| Enzyme CoQ10 | ubiquinone | 0.5 |
| 1,3-propanediol | propylene glycol | 0.5 |
| Other ingredients | | 0.1 |

### Example 5

Example 5

| Ingredient | INCI Name | % of formulation |
|---|---|---|
| Water | aqua | 86.9 |
| Hyaluronic acid | sodium hyaluronate | 2.0 |
| Growth Media | human stem cell growth | 4.0 |
| | media | |
| Trans-resveratrol | resveratrol as a part of | 0.25 |
| cyclodextrin* complex | complex | |
| Quercetin cyclodextrin* | quercetin as a part of | 0.25 |
| complex | complex | |
| Fisetin cyclodextrin* | Fisetin as a part of | 0.25 |
| complex | complex | |
| Ferulic acid cyclodextrin* | ferulic acid as a part of | 0.25 |
| complex | complex | |
| 2-HP-β-cyclodextrin | Total part of cyclodextrin | 6.0 |
| | complexes | |
| Other ingredients | | 0.1 |

*Cyclodextrin- 2-HP-β-cyclodextrin

### Human Testing Results

A 62 year old Caucasian female (11 years post-menopause) was diagnosed with atrophic vaginitis, vaginal atrophy (as confirmed by optical coherence tomography) and vaginal dryness secondary to menopause on history and gynecological examination. The patient complained of painful sexual intercourse. She was prescribed hormonal cream. The patient on reading the side effects to the hormonal preparation declined to use the cream and started using over-the-counter vaginal lubricants. The patient reported that the several personal lubricants produced a distinct burning sensation on application. Pain on the initiation of intercourse remained but was partially diminished with the vaginal lubrication products. The patient reported that the over-the-counter personal lubricants did not alleviate painful intercourse and had the side effects of tissue burning. Testing of the individual formulations was conducted sequentially with prolonged non-treatment intervals as described.

### Example 1 Testing

The patient started a course of using the subject light delivery invention 2 times a week with the concurrent application of 0.5 ml sample formula Example 1 (1.25% Hyaluronic acid) 1 time daily at bedtime. The patient continued this regime for 28 days. After 7 days of use, the patient engaged in sexual intercourse; the patent reported a greatly reduced amount of pain and a general feeling of a more natural feeling within the vagina. At 14 days the patent engaged in sexual intercourse and found there were no symptoms of pain. The patent reported the total absence of pain on at 14, 21 and 28 days after the start of treatment. At 30 days, the dosage was increased to 2 ml. The patient complained that the majority of the administered dose leaked out of the vagina immediately after application. Treatment was discontinued after 30 days. The patient did not report any adverse side effects or irritation or burning. The patient did state that there was a general soothing feeling on application.

At 7 days after treatment discontinuation the patient reported a slight pain of sexual intercourse. At 14 days the patent reported about the same amount of pain prior to treatment. There was no treatment for the next 35 days.

### Example 2 Testing

A second course of treatment was started using the formulation Example 2 (4% Hyaluronic acid). The patient started a course of using the subject light delivery invention 2× a week with the concurrent application of 2 ml sample formula Example 2 1 time daily at bedtime. After 7 days of use, the patient engaged in sexual intercourse; the patent reported the absence of pain during sexual intercourse. The patient reported the total absence of pain on at 14, 21 and 28 days after the start of treatment. The patient reported that the formulation did not leak after administration. Treatment was stopped after 28 days of use.

The patient did not report any adverse side effects or irritation or burning. The patient did repeat that there was a generally soothing and natural feeling on the application and continued use.

At 7 days after treatment discontinuation of the second treatment regimen, the patient reported no pain of sexual intercourse. At 14 days after treatment discontinuation, the patent reported a slight pain of sexual intercourse. At 28 days, the patent reported about the same amount of pain prior to treatment. There was no treatment for the next 28 days.

### Example 3 Testing

A third course of treatment was started using the formulation Example 3 (4% Hyaluronic acid). The patient started a course of using the subject light delivery invention 2 times a week with the concurrent application of 2 ml sample formula Example 2 1× daily at bedtime. After 7 days of use, the patient engaged in sexual intercourse; the patient reported the absence of pain during sexual intercourse. The patient reported the total absence of pain on at 14, 21 and 28 days after the start of treatment. The patient reported that the formulation did not leak after administration. At 28 days, light treatment was reduced to 1 time/week with concurrent application of Example 3 formulation while the patent continued to administer 2 ml of Example 3 formulation every 2-3 days at bedtime. The patient has continued with this regime for over 210 days with no adverse side effects. The patient engaged in regular sexual intercourse with no pain. On gynecologic examination, the same initial treating gynecologist reported an absence of obvious signs atrophic vaginitis, vaginal atrophy (as confirmed by optical coherence tomography) and vaginal dryness as well as a general improvement in the health of the vagina.

### Example 4 Testing

A fourth course of treatment was started using the formulation Example 4 (4% Hyaluronic acid and 0.5% resveratrol). The patient started a course of using the subject light delivery invention 2 times a week with the concurrent application of 2 ml sample formula Example 4 1 time daily at bedtime. After 7 days of use, the patient engaged in sexual intercourse; the patent reported the absence of pain during sexual intercourse. The patient reported the total absence of pain on at 14, 21 and 28 days after the start of treatment. The patient reported that the formulation did not leak after administration. At 28 days, light treatment was reduced to 1 time/week with concurrent application of Example 4 formulation while the patient continued to administer 2 ml of Example 4 formulation every 2-3 days at bedtime. The patient has continued with this regime for over 300 days with no adverse side effects. The patent engaged in regular sexual intercourse with no pain. On repeated gynecologic examination, the same initial treating gynecologist reported an absence of obvious signs atrophic vaginitis, vaginal atrophy, and vaginal dryness as well as a general improvement in the health of the vagina in this patient.

### Example 5 Testing

A fifth course of treatment was started using the formulation Example 5 (2% Hyaluronic acid, 4% growth media, 0.25% resveratrol, 0.25% quercetin, 0.25% fisetin, 0.25% ferulic acid). The patient started a course of using the subject light delivery invention 2 times a week with the concurrent application of 2 ml sample formula Example 5 1 time daily at bedtime. After 7 days of use, the patient engaged in sexual intercourse; the patent reported the absence of pain during sexual intercourse. The patient reported the total absence of pain on at 14, 21 and 28 days after the start of treatment. The patient reported that the formulation did not leak after administration. At 28 days, light treatment was reduced to 1 time/week with concurrent application of Example 5 formulation while the patient continued to administer 2 ml of Example 5 formulation every 2-3 days at bedtime. The patient has continued with this regime for over 100 days with no adverse side effects. The patent engaged in regular sexual intercourse with no pain. On repeated gynecologic examination, the same initial treating gynecologist reported an absence of obvious signs atrophic vaginitis, vaginal atrophy, and vaginal dryness as well as a general improvement in the health of the vagina in this patient.

There were no adverse side effects from any of the topical formulations tested or from the combination use of the therapeutic compositions with concurrent light irradiation using the intravaginal light device.

What is claimed is:

1. A method for intravaginal trans-membrane interstitial delivery of a biologically active composition, the method comprising illuminating, with a pulsed light having a selected wavelength, selected pulse rate and selected duty cycle, a formulation applied to the non-keratinized cellular surface of a mucus membrane within the vagina and onto vaginal tissues of a human adult female, in order to allow the biologically active substance in the formulation to permeate through said cellular surface of the mucus membrane, wherein the formulation comprises the biologically active substance, and wherein the pulsed light originates from a light source comprised in a vaginally insertable light irradiating device inserted in the vagina of the human adult female, thereby effecting light facilitated intra-vulvovaginal, intravaginal, intramucosal, and/or interstitial deposition of the biologically active substance in interstitial concentrations exceeding the achievable interstitial concentrations attainable by passive concentration gradient topical application.

2. The method of claim 1, wherein the method provides targeted delivery of said biologically active composition into vaginal tissues while preventing damages to vaginal tissues, and further preventing unpredictable effects resulting from non-targeted systemic absorption of said biologically active composition.

3. A light irradiating vaginal dilating device, comprising a longitudinally progressively increasing diameter to affect gradual vaginal dilation, and a source a pulsed light having a selected wavelength, selected pulse rate and selected duty cycle to provide for intravaginal trans-membrane interstitial delivery of a biologically active composition, when the device is inserted in the vagina of an adult human female.

4. The device of claim 3, comprising an inflatable dilating sheath to effect progressive dilation without causing tissue ischemia.

5. The device of claim 4, which is capable of providing increasing dilation pulsatile pressure within an atrophied vagina to affect dilation and increase tissue flexibility and compliance.

6. An intravaginal photokinetic delivery device, comprising:
a. a housing of an optically clear material, of the size and shape (e.g., a cylindrical shape with a domed end) suitable for insertion into the vagina of an adult human female;
b. a cavity within said housing to accommodate an electronic package, said electronic package comprising:
i) an electronic pulse generator that provides pulsed electronic drive current to an LED;
ii) an electric circuitry that allows for independent control of at least one (e.g., 2, 3, 4, or more) group(s) of LEDs, at a selected wavelength for each group of LEDs;
iii) a battery pack that provides power source for electronic pulse generator, the electric circuitry, and/or the LEDs.

7. The device of claim 6, wherein the optically clear material comprises clear medical grade silicone, polymethyl methacrylate, polycarbonate, polyethylene terephthalate, hardened glass, crystal glass, or quartz.

8. The device of claim 6 or 7, wherein the size of the housing is variable to accommodate differences in vagina size and/or vaginal opening.

9. The device of any one of claims 6-8, wherein the size of the housing is larger than an intended vaginal target site to allow for smooth insertion and a desired degree of vaginal stretching.

10. The device of any one of claims 6-9, wherein the size of the housing allows for placement of the housing within the vagina without causing pain, while being sufficiently large to maintain the housing in place without ejection from its own weight by gravity alone.

11. The device of any one of claims 6-10, wherein the housing comprises a cone-shaped distal tip with a longitudinally progressively enlarged diameter along a shaft towards the proximal end, for insertion into the vagina of an adult human female.

12. The device of claim 11, wherein the proximal end of the housing comprises a handle area angled away from the shaft to allow ergonomic ease of use when the shaft is inserted into the vagina.

13. The device of claim 12, wherein the electronic package is housed within the handle area.

14. The device of any one of claims 6-13, wherein the selected wavelength for each group of LEDs is different or the same among the different groups of LEDs.

15. The device of any one of claims 6-14, wherein the battery pack is a rechargeable battery pack that can be recharged with, e.g., a magnetic induction system, which optionally has an integral charging control circuitry.

16. The device of any one of claims 6-15, wherein the electric circuitry is controlled by a mechanical switch, or a magnetically-operated switch, or a radio frequency controlled switch, wherein the switch is optionally sealed with a flexible cover.

17. The device of any one of claims 6-15, wherein the electronic package is imbedded, encapsulated, and/or potted within the housing, optionally with optically clear epoxy or an electronic embedding substance.

18. The device of any one of claims 6-17, wherein the material for the house is substantially free of optical absorption in the wavelengths of the lights emitted by the LEDs.

19. The device of any one of claims 6-18, wherein the housing comprises mechanical etching, laser etching, or cast in place structure that scatters and/or redirects LED lights without causing light loss while directing the LED light out of the device.

20. The device of any one of claims 6-19, further comprising attachment points for affixing a retrieving structure, such as a flexible retrieval string loop, to retrieve the device after use.

21. The device of claim 20, further comprising the retrieving structure affixed to the attachment points on the housing.

22. The device of claim 21, wherein the retrieving structure is a flexible monofilament material or a coated multi-strand string structure.

23. The device of any one of claims 20-22, wherein the retrieving structure serves as an antenna for external communication to a device-controlling program or application.

24. The device of any one of claims 20-22, wherein the retrieving structure is a flexible or semirigid stem configured to aid in vaginal placement of the device, such that the housing and the stem are in close proximity to each other and to the urethra opening to provide direct light irradiation and vibration to the urethral opening to treat urinary incontinence.

25. The device of claim 24, wherein the stem has LEDs positioned to irradiate external genitalia to initiate increased blood flow to the tissues for enhanced sexual wellness.

26. The device of claim 25, wherein the distal end of the stem has a progressive pressure switch to operate the LEDs, and/or the vibration motor (if present).

27. The device of any one of claims 24-26, further comprising an antenna on the stem for remote controlling and information transfer to an external electronic device.

28. The device of any one of claims 24-27, further comprising a vaginal pressure sensor within the stem or the housing to track vaginal exercise and vaginal contraction.

29. The device of any one of claims 6-28, wherein the surface of the cavity or the electronic package is coated with a reflective surface (e.g., a painted polishing mirror) directing the LED light away from the electronic package.

30. The device of claim 29, wherein the LEDs are mounted on top of the reflective surface, with light output directed away from the electronics package.

31. The device of any one of claims 6-30, further comprising a vibration generating motor.

32. A charging housing for charging the device of any one of claims 6-31, comprising an integral electrical induction coil, positioned within the charging housing such that, when the charging housing is in charging mode, contacts a charging electrode electronically connected to the battery pack of the device.

33. The charging housing of claim 32, which is a box with a lid, and the charging mode is engaged when the lid is closed with the device being inside the box.

34. A method for intravaginal trans-membrane interstitial delivery of a vaginal tissue conditioning and moisturizing biologically active composition, the method comprising:
1) applying the biologically active composition to the non-keratinized cellular surface of a mucus membrane within the vagina and onto vaginal tissues of an adult human female;
2) inserting an intravaginal light device of any one of claim 6-33 to illuminate said biologically active composition applied to the non-keratinized surface of the vaginal tissue with a pulsed light having a selected wavelength, selected pulse rate and selected duty cycle; and,
3) allowing the biologically active composition to permeate through said cellular surface of the mucus membrane to provide tissue conditioning moisturization and compliance,
thereby effecting light facilitated intra-vulvovaginal, intravaginal, intramucosal, and/or interstitial deposition of the biologically active composition in interstitial concentrations exceeding the achievable interstitial concentrations attainable by passive concentration gradient topical application.
